(19) United States
(12) Patent Application Publication
(10) Pub. No.: US 2026/0157849 A1
(43) Pub. Date: Jun. 11, 2026

(54) Leaflet Modification for Graduated Reduction in Valve Regurgitation

(71) Applicant: St. Jude Medical, Cardiology Division, Inc., St. Paul, MN (US)

(72) Inventor: Stephanie Kwong, Minnetonka, MN (US)

(73) Assignee: St. Jude Medical, Cardiology Division, Inc., St. Paul, MN (US)

(21) Appl. No.: 19/317,874

(22) Filed: Sep. 3, 2025

Related U.S. Application Data
(60) Provisional application No. 63/730,727, filed on Dec. 11, 2024.

(51) Int. Cl. A61F 2/24

(52) U.S. Cl. CPC A61F 2/2412; A61F 2210/0004; A61F 2220/005

(57) ABSTRACT

A prosthetic heart valve includes a frame and a plurality of prosthetic leaflets mounted within the frame. A first one of the plurality of prosthetic leaflets includes a fixation mechanism. The fixation mechanism may be configured to increase a maximum dimension of one of the plurality of leaflets over a period of time in situ. The fixation mechanism may alternatively or additionally restrict mobility of one or more of the prosthetic leaflets to induce regurgitation when the prosthetic heart valve is implanted. The fixation mechanism may transition over time so that the amount of intentional regurgitation reduces over time, including reducing to substantially zero regurgitation, or any other intentionally designed level of regurgitation. The intentional regurgitation may allow the heart to safely acclimate to new pressure dynamics over time after the implantation.

### CROSS-REFERENCE TO RELATED APPLICATIONS

This application claims priority to U.S. Provisional Patent Application No. 63/730,727, filed Dec. 11, 2024, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The human heart includes four heart valves, including the aortic valve, the mitral valve (also known as the bicuspid valve or the left atrioventricular valve), the pulmonary valve, and the tricuspid valve (also known as the right atrioventricular valve). Although prosthetic heart valves intended for use in replacing the various native heart valves may typically have design elements in common, each native heart valve has unique anatomical considerations that may need to be considered to design an optimal prosthetic heart valve. For example, the mitral valve and the tricuspid valve are typically significantly larger than the aortic valve and the pulmonary valve, and thus prosthetic heart valves designed to replace the mitral valve or the tricuspid valve typically need to have larger frames than those designed to replace the aortic valve or the pulmonary valve. Pressure generated by the left ventricle is typically significantly larger than pressure generated by the right ventricle, and thus prosthetic heart valves designed to replace the mitral valve typically need more substantial anchoring mechanisms than prosthetic heart valves designed to replace the tricuspid valve. The present disclosure addresses problems and limitations associated with the related art.

### BRIEF SUMMARY OF THE DISCLOSURE

Prosthetic heart valves may be used to replace the functionality of a failing heart valve. One example of a failing heart valve is one that allows for blood to flow backward through the heart valve in the retrograde direction. Typically, prosthetic heart valves are designed to mostly or entirely eliminate any regurgitation across the prosthetic heart valve. However, in some circumstances, implanting a prosthetic heart valve and immediately resolving the regurgitation may be undesirable. For example, in patients with severe tricuspid valve regurgitation, the right ventricle wall may be particularly thin. Immediately resolving regurgitation could create a situation in which the right ventricle is not able to handle the new pressures, which could lead to problems such as decompensated heart failure. At least some examples of prosthetic heart valves described herein may provide for a level of intentional regurgitation, which in some cases reduces over time, which may provide benefits such as providing heart tissue time to acclimate to new hemodynamics following a prosthetic heart valve implantation.

According to an aspect of the disclosure, a prosthetic heart valve includes a frame and a plurality of prosthetic leaflets mounted within the frame. A first one of the plurality of prosthetic leaflets includes a fixation mechanism applied to the first one of the plurality of prosthetic leaflets, the fixation mechanism configured to transition from an initial condition at an initial time point to a subsequent condition at a subsequent time point after implantation. When the fixation mechanism is in the initial condition, the fixation mechanism is configured to restrict mobility of the first one of the plurality of prosthetic leaflets to induce a first amount of regurgitation of blood through the plurality of prosthetic leaflets. When the fixation mechanism is in the subsequent condition, the fixation mechanism is configured to induce a second amount of regurgitation of blood through the plurality of prosthetic leaflets. The second amount of regurgitation is less than the first amount of regurgitation. The fixation mechanism may be configured to dissolve, degrade, absorb or otherwise release between the first time point and the subsequent time point after implantation to transition the fixation mechanism from the initial condition to the subsequent condition. The fixation mechanism may be an adhesive. The fixation mechanism may be a binding-type mechanism, agent, or material. The second amount of regurgitation may be between about 0 mL and about 5 mL per heartbeat. The first one of the plurality of prosthetic leaflets may include a terminal free edge and an attached edge, with a portion of the terminal free edge being folded over and attached, via the fixation mechanism, to a remaining portion of the first one of the plurality of prosthetic leaflets to form a modified free edge portion. In the initial condition of the fixation mechanism, the first one of the plurality of prosthetic leaflets may have an initial effective axial height between the attached edge and the modified free edge portion, and in the subsequent condition of the fixation mechanism, the terminal free edge may no longer be folded over and attached to the remaining portion of the first one of the plurality of prosthetic leaflets such that the first one of the plurality of prosthetic leaflets has a subsequent effective axial height between the attached edge and the terminal free edge, the subsequent effective axial height being greater than the initial effective axial height.

In the initial condition of the fixation mechanism, the first one of the plurality of prosthetic leaflets may include a gathered portion to which the fixation mechanism is applied to maintain the gathered portion in a gathered condition. In the initial condition of the fixation mechanism, the gathered portion may extend generally orthogonal to a direction of blood flow through the prosthetic heart valve. In the initial condition of the fixation mechanism, the first one of the plurality of prosthetic leaflets may have an initial effective axial height between an attached edge of the first one of the plurality of prosthetic leaflets and a free edge of the first one of the plurality of prosthetic leaflets, and in the subsequent condition of the fixation mechanism, the first one of the plurality of prosthetic leaflets may have a subsequent effective axial height between the attached edge and the free edge, the subsequent effective axial height being greater than the initial effective axial height. In the initial condition of the fixation mechanism, the gathered portion may extend generally parallel to a direction of blood flow through the prosthetic heart valve. In the initial condition of the fixation mechanism, a free edge of the first one of the plurality of prosthetic leaflets may have an initial effective length and an initial tautness, and in the subsequent condition of the fixation mechanism, the free edge may have a subsequent effective length and a subsequent tautness, the subsequent effective length being greater than the initial effective length, the initial tautness being greater than the subsequent tautness. In the initial condition of the fixation mechanism, a portion of the first one of the plurality of prosthetic leaflets may be attached, via the fixation mechanism, to (i) the frame and/or (ii) a skirt coupled to the frame. In the initial condition of the fixation mechanism, the fixation mechanism may prevent the portion of the first one of the plurality of prosthetic leaflets from moving toward remaining ones of the plurality of prosthetic leaflets, and in the subsequent condition of the fixation mechanism, the fixation mechanism may not prevent the portion of the first one of the plurality of prosthetic leaflets from moving toward remaining ones of the plurality of prosthetic leaflets. The portion of the first one of the plurality of prosthetic leaflets may be a portion of a free edge of the first one of the plurality of prosthetic leaflets.

According to another aspect of the disclosure, a prosthetic heart valve includes a frame and a plurality of prosthetic leaflets mounted within the frame. A first one of the plurality of prosthetic leaflets includes a first fixation mechanism having (i) an initial state in which the first fixation mechanism prevents the first one of the plurality of prosthetic leaflets from fully coapting with remaining ones of the plurality of prosthetic leaflets so that regurgitation of blood through the plurality of prosthetic leaflets is maintained in the initial state, and (ii) a subsequent state in which the first fixation mechanism does not prevent the first one of the plurality of prosthetic leaflets from fully coapting with the remaining ones of the plurality of prosthetic leaflets. The first one of the plurality of prosthetic leaflets includes a second fixation mechanism, the second fixation mechanism having (i) an initial state in which the second fixation mechanism prevents the first one of the plurality of prosthetic leaflets from fully coapting with remaining ones of the plurality of prosthetic leaflets so that regurgitation of blood through the plurality of prosthetic leaflets is maintained in the initial state, and (ii) a subsequent state in which the second fixation mechanism does not prevent the first one of the plurality of prosthetic leaflets from fully coapting with the remaining ones of the plurality of prosthetic leaflets. When the first fixation mechanism and the second fixation mechanism are both in their respective subsequent states, regurgitation of blood through the plurality of prosthetic leaflets is less than when the first fixation mechanism and the second mechanism are both in their respective initial states. The first fixation mechanism may be configured to dissolve, degrade, absorb or otherwise release over time to transition the first fixation mechanism from the initial state to the subsequent state, and the second fixation mechanism may be configured to dissolve, degrade, absorb or otherwise release over time to transition the second fixation mechanism from the initial state to the subsequent state. The first fixation mechanism may be configured to transition from the initial state to the subsequent state at a first time after implantation of the prosthetic heart valve, and the second fixation mechanism may be configured to transition from the initial state to the subsequent state at a second time after implantation of the prosthetic heart valve, the first time being different than the second time. The first time may be about one month, and the second time may be about three months or about six months. The first time may be about one month or about three months, and the second time may be about six months.

According to a further aspect of the disclosure, a method of implanting a prosthetic heart valve includes implanting a prosthetic heart valve into a native heart valve annulus of a patient, the prosthetic heart valve including a frame and a plurality of prosthetic leaflets mounted within the frame. During an initial time frame after implanting the prosthetic heart valve, intentional regurgitation of blood is allowed through the plurality of prosthetic leaflets, the regurgitation resulting from a fixation mechanism of a first one of the plurality of prosthetic leaflets being in an initial state in which the fixation mechanism prevents the first one of the plurality of prosthetic leaflets from fully coapting with remaining ones of the plurality of prosthetic leaflets. After the initial time frame, the fixation mechanism transitions to a subsequent state in which the first one of the plurality of prosthetic leaflets coapts with remaining ones of the plurality of prosthetic leaflets to reduce an amount of the intentional regurgitation of blood through the plurality of prosthetic leaflets compared to the initial time frame. The fixation mechanism may dissolve, degrade, absorb or otherwise release after the initial time frame. The fixation mechanism may be an adhesive. The fixation mechanism may be a binding-type mechanism, agent, or material. After the initial time frame, the amount of intentional regurgitation of blood through the plurality of prosthetic leaflets may be between about 0 mL and about 5 mL per heartbeat.

According to another aspect of the disclosure, a prosthetic heart valve includes a frame and a plurality of prosthetic leaflets mounted within the frame. A first one of the plurality of prosthetic leaflets includes a fixation mechanism. The fixation mechanism is configured to increase a maximum dimension of one of the plurality of leaflets over a period of time in situ. The maximum dimension may be, for example, an effective axial height, and effective length (e.g. circumferential length or edge-to-edge length), or an effective surface area. One or more of the features and characteristics of the above paragraphs in the summary may also be included in this aspect of the disclosure.

### BRIEF DESCRIPTION OF THE DRAWINGS

FIG. 1 is a side view of a small-waisted prosthetic heart valve in an expanded, deployed condition.

FIG. 2 is a view of a cut pattern of a frame of the prosthetic heart valve of FIG. 1.

FIG. 3 is an enlarged view of the commissure attachment feature of the frame of FIGS. 1-2.

FIG. 4 is a view of a cut pattern of the commissure support structure of FIG. 1

FIG. 5 is an enlarged view of a portion of the cut pattern of FIG. 4.

FIG. 6 is a side view of a large-waisted prosthetic heart valve in an expanded condition.

FIG. 7 is a view of a cut pattern of a frame of the prosthetic heart valve of FIG. 6.

FIG. 8 is a perspective view of the frame of the prosthetic heart valve of FIG. 6, isolated from other components of the prosthetic heart valve, in an expanded condition.

FIG. 9 is a schematic representation of a leaflet of the prosthetic heart valve of FIG. 6.

FIG. 10A is a front view of a prosthetic leaflet, as if laid out on a table, according to an aspect of the disclosure.

FIG. 10B is a side view of a portion of the prosthetic leaflet of FIG. 10A in one example of an initial use condition.

FIG. 10C is a side view of the portion of the prosthetic leaflet of FIG. 10B in one example of a final use condition.

FIGS. 11A-1 and 11A-2 are front and side views, respectively, of a prosthetic leaflet in another example of an initial use condition.

FIGS. 11B-1 and 11B-2 are front and side views, respectively, of the prosthetic leaflet of FIGS. 11A-1 and 11A-2 in one example of an intermediate use condition.

FIGS. 11C-1 and 11C-2 are front and side views, respectively, of the prosthetic leaflet of FIGS. 11A-1 and 11A-2 in one example of a final use condition.

FIGS. 12A and 12B are front and top views, respectively, of a prosthetic leaflet in an example of an initial use condition.

FIGS. 12C and 12D are front and top views, respectively, of the prosthetic leaflet of FIGS. 12A and 12B in an example of a final use condition.

FIG. 13A is a schematic top view of a prosthetic heart valve in one example of an initial use condition during a coaptation phase of the prosthetic heart valve.

FIG. 13B is a schematic top view of the prosthetic heart valve of FIG. 13A in one example of a final use condition during the coaptation phase of the prosthetic heart valve.

FIG. 14A is a front view of a prosthetic leaflet, as if laid out on a table, according to an aspect of the disclosure.

FIG. 14B is a front view of a prosthetic leaflet, as if laid out on a table, according to an aspect of the disclosure.

FIG. 14C is a front view of a prosthetic leaflet, as if laid out on a table, according to an aspect of the disclosure.

FIG. 14D is a front view of a prosthetic leaflet, as if laid out on a table, according to an aspect of the disclosure.

FIG. 15 is a flow chart showing example steps of method of manufacturing and/or using a prosthetic heart valve.

FIG. 16 is a flow chart showing another example steps of a method of manufacturing and/or using a prosthetic heart valve.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure may apply with similar force to other prosthetic valve replacements. Thus, unless otherwise expressly specified, the embodiments described herein may be used for replacing any native heart valve (with or without additional modifications specific to the heart valve being replaced), even if a particular embodiment may be more suited for replacing the native tricuspid valve than another native heart valve.

As noted above, each native heart valve has unique anatomical considerations that may need to be accounted for when designing an optimal replacement. For example, the wall of the left ventricle in many patients is thicker than the wall of the right ventricle. Further, a patient with severe or torrential tricuspid valve regurgitation may have an even thinner right ventricle wall due to dilation from heart failure. While prosthetic heart valves are typically designed to immediately resolve regurgitation (e.g. blood flowing in the retrograde direction through the valve), counterintuitively, it may be preferable that a prosthetic tricuspid valve does not immediately resolve all tricuspid regurgitation. In fact, in patients with severe or torrential tricuspid regurgitation, there may be a danger of transitioning between extreme tricuspid regurgitation to zero (or near zero) regurgitation in what is effectively an instantaneous change following implantation of a prosthetic tricuspid valve. This potential danger lies, at least in part, in the fact that the flow dynamics change nearly instantaneously, while the heart muscle cannot acclimate to the new flow dynamics instantaneously. After tricuspid regurgitation is resolved, pressures within the right ventricle may increase. If the muscle of the heart, and in particular the right ventricle, is not able to appropriately acclimate (for example by strengthening and thickening over time), the patient may be at risk of right ventricular rupture, further dilation, and/or decompensated heart failure.

As is explained in greater detail below, in order to compensate for the risks or instantaneously resolving tricuspid regurgitation, prosthetic tricuspid valves may be designed so that the prosthetic leaflets intentionally allow for some amount of regurgitation so as to not instantaneously resolve the tricuspid regurgitation upon implantation. In some examples described in greater detail below, the amount of intentional regurgitation may change over time, so that regurgitation decreases over time while the heart muscle is able to strengthen and acclimate to the new flow dynamics over a longer time period. In other words, by the time the intentional regurgitation decreases to zero (or substantially zero or any other prescribed or otherwise intentionally designed level) regurgitation, the heart tissue will have already acclimated to the new hemodynamics, so that upon complete (or substantially complete) elimination of regurgitation (or reduction to any otherwise intentionally designed level of regurgitation), the heart tissue has already gotten stronger and is more “ready” for the change in hemodynamics that occurs upon complete (or substantially complete) elimination of regurgitation (or reduction to any otherwise intentionally designed level of regurgitation). It should be understood that tricuspid valve regurgitation, as a general matter, is less symptomatic and better tolerated than mitral valve regurgitation. Thus, while designing intentional regurgitation into a prosthetic mitral valve may be dangerous for a patient, the same feature in a prosthetic tricuspid valve may not only be tolerated by a patient, but may actually result in better overall outcomes. Notwithstanding the above, in some situations, the embodiments described below in connection with intentionally regurgitant prosthetic heart valve leaflets may have use in prosthetic heart valves other than in prosthetic tricuspid valves, including prosthetic mitral, aortic, or pulmonary valves.

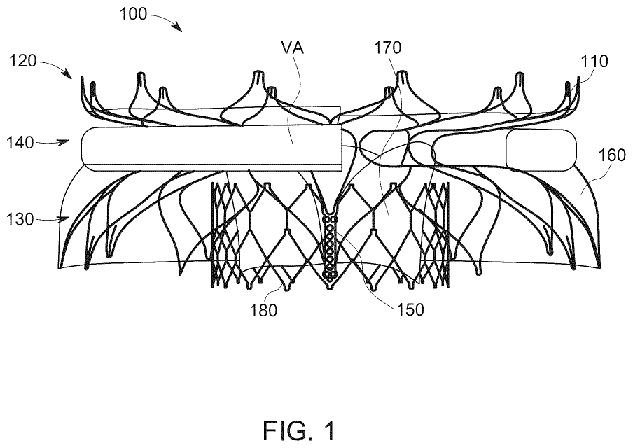

FIG. 1 is a side view of a prosthetic heart valve 100, in particular a prosthetic atrioventricular valve, shown in an expanded, implanted condition. Generally, prosthetic heart valve 100 includes a frame 110, a skirt 160, prosthetic leaflets 170, and a support ring 180. In FIG. 1, the skirt 160 is shown as partially transparent so that internal components of the prosthetic heart valve 100 are visible. Furthermore, a representation of a portion of a native heart valve annulus VA is shown in FIG. 1 with one quarter of the circumference of the valve annulus VA cut-away to better illustrate how a waist of the frame 110, described in greater detail below, engages the valve annulus VA.

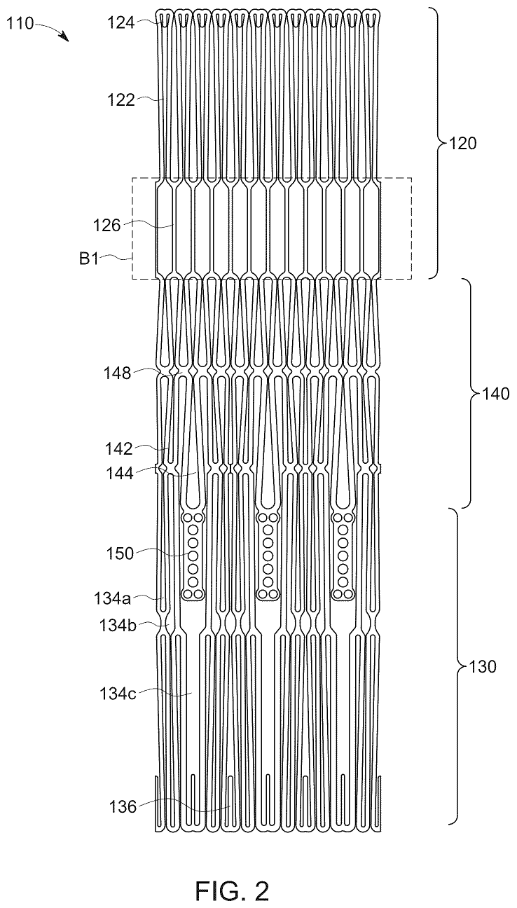

FIG. 2 shows a cut pattern of frame 110, as if the frame 110 were cut longitudinally and laid on a table. Referring to FIGS. 1 and 2, frame 110 may include an atrial flange 120 (which may alternately be referred to as an atrial disk or anchor), a ventricular flange 130 (which may alternately be referred to as a ventricular disk or anchor), and a central frame portion 140 (which may be referred to as a central waist). The frame 100 may also include a plurality of commissure attachment features (“CAFs”) 150 for use in coupling the prosthetic valve leaflets 170 to the frame 110.

Frame 110 may be formed of a biocompatible shape memory or superelastic material. One suitable example of this frame material is a nickel-titanium alloy, such as nitinol. However, other materials may be suitable. In one example, frame 110 may be formed by laser cutting a hollow tube of nitinol, and then shape-setting the frame 110 to the desired shape, for example by heat treatment. With this configuration, the frame 110 may take the set shape, such as that shown in FIG. 1, in the absence of applied forces. To deliver the prosthetic heart valve 100, the prosthetic heart valve 100 may be collapsed to a small diameter and positioned within a delivery catheter to be passed intravascularly through the patient into the patient's heart.

Referring to FIGS. 1-2, the frame 110 may be formed with a plurality of rows of generally diamond-shaped cells. In the illustrated example, the atrial flange 120 includes an inflow row of cells 122, which may include a total of twelve cells. A pin 124 may be formed at the inflow apex of one, some, or each cell 122, the pin extending a short distance in the outflow direction to a free end. Each pin 124 may be sized and shaped so that a suture loop of the delivery device may slip over the pin 124, keeping the frame 110 connected to the delivery device during delivery and deployment. Upon deployment of the prosthetic heart valve 100, each suture loop may be pushed forward or distally to disengage with the corresponding pins 124 to fully decouple the prosthetic heart valve 100 from the delivery device. Similar pins and suture loops are described in more detail in U.S. Pat. No. 10,874,512, the disclosure of which is hereby incorporated by reference herein. The atrial cells 122 may terminate, at their outflow ends, at an inflection point 148. When the frame 110 is shape-set to the desired shape, which may be generally similar to that shown in FIG. 1, the inflection points 148 may define the smallest diameter of the center portion 140. It should be understood that the term “inflection point” is not necessarily used according to its mathematical definition, but rather references the point at which the frame 110 changes from decreasing diameter to increasing diameter.

A plurality of transition cells 142, which may be generally diamond-shaped, may be positioned in a row that is adjacent to the atrial cells 122 in the outflow direction. Transition cells 142 may include an inflow portion on the inflow side of center portion 140 and an outflow portion on the outflow side of center portion 140. In some examples, the transition cells 142 may be axially centered about the inflection point 148. The row of transition cells 142 may include three enlarged transition cells 144 (or more or fewer than three depending on the number of prosthetic leaflets included in the prosthetic heart valve 100) that each terminate in a commissure attachment feature (“CAF”) 150. The enlarged transition cells 144 may be positioned at substantially equal circumferential intervals around the frame 110. As best shown in FIG. 2, the sides of the atrial cells 122 (which may extend to the inflow apex of the transition cells 142 and the enlarged transition cells 144) may include elongated beams 126. These elongated beams 126 may provide additional flexibility to the atrial flange 120 (which may be referred to as the atrial disk). For example, depending on the numbers of cells included in the atrial portion, and the desired diameter that the atrial portion will span, the length of the beams 126 may be adjusted. As the desired diameter of the atrial portion 120 increases, the length (in the axial direction) of the diamond-shaped cells that form the atrial portion 120 may need to increase if a particular opening angle (e.g., about 90 degrees) of the diamond-shaped cells is desired. As the axial length of the diamond-shaped cells increases in the differently-sized valve frames, the beams 126 may correspondingly increase or decrease in length. However, in some embodiments, the beams 126 may be omitted and the atrial row of cells 122 may all be “full” diamond-shaped cells.

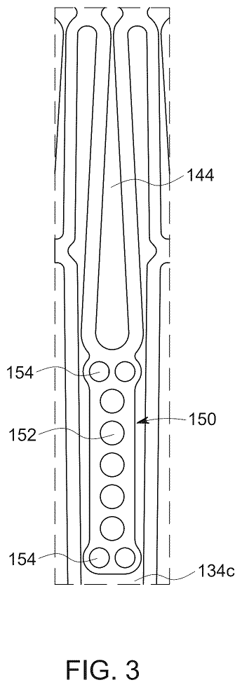

Each CAF 150 may serve as an attachment point to the prosthetic leaflets 170. For example, each CAF 150 may include a plurality of holes, and sutures may be used to couple adjacent pairs of leaflets to the CAFs 150 via the holes therein. CAFs 150 are described in greater detail below in connection with FIG. 3.

The portion of the frame 110 in the outflow direction of the inflection point 148 may include a plurality of ventricular cells. For example, a group of first ventricular cells 134a which may be generally diamond-shaped cells, the inflow apex of which is an inflection point 148. A group of second ventricular cells 134b may extend to the outflow-most portion of the frame 110, the inflow apices of the second ventricular cells being connected to the outflow apices of the transition cells 142. Some, none, or all of the second ventricular cells 134b may include tines 136, described in greater detail below, that may act as frictional engagement members that frictionally engage native tissue for enhancing securement of the frame 110 within the native valve annulus. A group of third ventricular cells 134c may be positioned between certain pairs of second ventricular cells 134b, and may include struts that extend from the inflection point 148 to the terminal outflow end of the ventricular portion 130. Third ventricular cells 134c may be larger than the other ventricular cells and may be formed in part by the struts of enlarged transition cells 144 that terminate at CAFs 150. With this configuration, at least in the cut pattern shown in FIG. 2, the CAFs 150 may be thought of as either nested within third ventricular cells 134c or forming a boundary of third ventricular cells 134c.

In addition to tines 136 being positioned in some, none, or all of the second ventricular cells 134b, none, some, or all of the third ventricular cells 134c may include tines 136. In the embodiment of FIG. 2, only some of the second ventricular cells 134b include tines 136, such that each second ventricular cell 134b that includes a tine 136 includes a single tine 136 extending upward (in the inflow direction) from an outflow apex of the cell. All of the tines 136 may extend to a free tip that may have a sharp or blunt point, that is intended either to pierce tissue or to frictionally engage the tissue without piercing it. It should be understood that the number and positioning of the tines 136 may be different from those shown in FIGS. 1-2, and the specific number and positioning shown in FIGS. 1-2 is merely exemplary. It should also be understood that, although the tines 136 are shown in FIG. 1 as being generally within the plane of the ventricular cells, in use, the tines 136 may extend radially outward (and in some embodiments through the skirt 160) from the ventricular cells to be better situated for frictionally engaging tissue.

In the illustrated embodiments, the tines 136 may be connected at an outflow end of the tine, with the free tip being positioned at an inflow end of the tine. This directionality of tines, compared to the tines being connected at their inflow end and having free tips at their outflow ends, may allow for a smoother and easier deployment of the valve from the delivery catheter. In other words, as the valve begins to self-expand as it is released from the delivery catheter, the tines do not begin to expand until the entire tine is free of the delivery device. With the opposite orientation, the tines might otherwise begin to extend radially outwardly and into contact with the end of the delivery sheath, which might make deployment more difficult. However, it should be understood that the illustrated directionality of tines may make the loading process slightly more difficult compared to the opposite directionality. However, smooth and easy deployment is typically more important than smooth and easy loading, and the loading process can be highly controlled and is performed outside the patient, while the deployment process is performed inside the patient.

After forming the frame 110 by using the cut pattern shown in FIG. 2, or another generally similar cut pattern, the frame 110 may be shape-set, for example via heat treatment, to the desired shape. FIG. 1 illustrates one example of frame 110 that has a cut pattern similar to that shown in FIG. 2, after having been shape set and having been connected to a skirt 160, prosthetic leaflets 170, and a commissure support 180, described in greater detail below.

As can be seen in FIG. 1, when the frame 110 is in the expanded or deployed condition the bottom of the atrial portion 120 may be substantially straight with a slight upward angle, with the top half of the atrial portion 120 may flare upwardly so that the tips of the atrial cells 122 point generally in the inflow direction. The contours described above may be other than exactly described while still being suitable for use in the prosthetic heart valve 100.

Still referring to FIG. 1, the ventricular portion 130 may form a general “bell” shape with a more rounded and less flat contour compared to the atrial portion 120. The more gentle contour of the ventricular portion 130 may allow for the ventricular portion 130 to drape against the ventricle and apply only light pressure to assist in fixing or otherwise securing the prosthetic heart valve 100 to the native valve annulus. This light pressure or draping may be a first mechanism by which the prosthetic heart valve 100 achieves fixation within the native valve annulus.

The various tines 136 described above may be shape set so that the free ends of the tines 136 are positioned away from the surfaces defined by the cell in which the tine 136 is located. In other words, the tines 136 may be bent or shaped so that the tips are available to pierce tissue or to frictionally engage tissue without piercing to provide a second mechanism by which the prosthetic heart valve 100 achieves fixation within the native valve annulus. The tines 136 may be oriented at different angles to achieve different objectives. For example, in some embodiments, some or all of the tines 136 may be oriented or angled with the free ends pointing toward the atrial portion 120 at an acute angle relative to the longitudinal axis passing through the center of the prosthetic heart valve 100. Tines 136 pointing at an acute angle, compared to a right angle or an obtuse angle, may be less likely to perforate tissue at the native valve annulus. Patients that may need a prosthetic atrioventricular valve, particularly a prosthetic tricuspid valve, may be likely to have very thin medial walls in the ventricle, and acutely angled tines 136 may particularly reduce the likelihood of the medial wall getting perforated by the tines 136. There may be additional benefits to having an acutely angled tine 136 compared to tines 136 with larger angles (e.g., right angle or obtuse angle), relating to loading and deployment of the prosthetic heart valve 100. For example, if the tines 136 are more acutely angled, they may provide less resistance when the prosthetic heart valve 100 is loaded into, or deployed from, the delivery catheter. Less resistance may equate to a more manageable load, which—all else being equal—may allow for a smaller size delivery catheter to be used. However, this is just one option. Some or all of the tines 136 may instead be shape set to be oriented more laterally, for example a relatively large acute angle, or a right or obtuse angle, relative to the central longitudinal axis of the prosthetic heart valve 100. Although the tines 136 may be entirely optional, if the tines 136 are included, whether they are acutely or laterally oriented, the tines 136 may provide a second mechanism by which the prosthetic heart valve 100 achieves fixation within the native valve annulus.

FIG. 3 shows an enlarged view of one of the CAFs 150. CAF 150 may be coupled to two struts at the outflow end of an enlarged transition cell 144. As noted above, CAF 150 may either be thought of as forming a boundary of, or being nested within, ventricular cells 134c. Each CAF 150 may have a general beam-shape and may include a single column of eyelets 152, with the column of eyelets 152 including a horizontal or circumferential row of two eyelets 154 flanking the opposite ends of the column of eyelets 152. As a result, the CAF 150 may have a general “dog-bone” shape. The column of eyelets 152 may provide for multiple options for a stable attachment of the commissures of the prosthetic leaflets 170 to the CAF 150, and the general shape of the CAF 150 does not foreshorten as the frame 110 expands, as may occur with diamond-shaped features. The horizontal pairs of eyelets 154 may help provide additional stability of the commissure tissue sutured through the eyelets 154 in the axial or flow direction of the prosthetic heart valve 100.

Before describing the support member 180 in more detail, an exemplary sealing skirt 160 that may be used with the prosthetic heart valve 100 is described. Referring to FIG. 1, an outer sealing skirt 160 may be provided on the exterior of the frame 110. In the particular example shown in FIG. 1, the sealing skirt 160 may be a single piece of material (although in some embodiments it may be a multi-piece design), which may be formed as a knitted fabric or a woven fabric (e.g., polyethylene terephthalate (“PET”), polytetrafluoroethylene (“PTFE”), ultra-high molecular weight polyethylene (“UHMWPE”), polyester, or similar materials). In the illustrated embodiment of FIG. 1, the sealing skirt 160 may have an atrial skirt portion and a ventricular skirt portion. The atrial skirt portion may be coupled to the atrial portion 120 of the frame 110 with a relatively tight connection—for example via suturing along the struts of the atrial portion 120 of the frame 110. In some embodiments, including that shown in FIG. 1, the inflow edge of the atrial skirt portion may be positioned a spaced distance from the atrial tips of the atrial cells 122. For example, in some embodiments the atrial end of the frame 110 inflects towards the atrium, and thus outer sealing skirt 160 may be terminated a spaced distance from the atrial end so that there is not a thrombogenic profile on the inflow end of the frame 110. In other embodiments, the inflow edge of the atrial skirt portion may be positioned to align with or cover the atrial tips of the atrial cells 122. It should be understood that the various tines 136 may pierce through the sealing fabric 160 so that the free ends thereof are available for frictional engagement with the native tissue upon implantation.

Still referring to FIG. 1, the ventricular skirt portion may be more loosely connected to the ventricular portion 130 of the frame 110 than the atrial skirt portion is connected to the atrial portion 120. For example, the outflow edge of the ventricular skirt portion may be relatively tightly coupled to the outflow end of the ventricular portion 130 of the frame 110, but the connection of the sealing skirt 160 may be relatively loose between the central portion 140 and the terminal end of the ventricular portion 130 of the frame 110. With this configuration, during ventricular systole (e.g., as the ventricle contracts, the prosthetic leaflets 170 close, and the pressure in the ventricle is greater than the pressure in the atrium), the pressure differential causes the ventricular skirt portion to billow, inflate, or parachute open. As the ventricular skirt portion parachutes during ventricular systole, it may fill any gaps, crevices, or openings between the prosthetic heart valve 100 and the native valve annulus that might otherwise result in blood leaking around the outside of the prosthetic heart valve 100 back into the atrium (i.e., paravalvular “PV” leak).

Referring still to FIG. 1, the illustrated configuration of frame 110 may provide a levering effect that may further assist with sealing against PV leak. For example, when the frame 110 is in the expanded or deployed state shown in FIG. 1, deformation of the ventricular portion 130 may tend to lever the atrial portion 120 toward the ventricular portion 130. Thus, as the ventricular skirt portion inflates or parachutes during ventricular systole, which may cause the ventricular portion 130 of the frame 110 to slightly deform, the atrial portion 120 of the frame 110 may be lightly pulled downward against the atrial side of the native valve annulus. This “sandwiching” action may further seal against any PV leak, and may also mitigate potential embolization. For example, particularly in the low flow environment of the right heart, any gaps or spaces left between the prosthetic heart valve 100 and the native anatomy may create a thrombus risk zone. The above-described levering or sandwiching effect may reduce or eliminate any such gaps or spaces, thus reducing the risk of thrombus formation. In one particular example, patients may have a pronounced septal bump, and some patients may have in particular a septal bump in the right ventricle that overhands the tricuspid valve annulus. This anatomy may be an exclusion criterion for a transcatheter prosthetic tricuspid valve replacement. However, the sandwiching or levering effect described above may allow for prosthetic heart valve 100 to be implanted into patients who have relatively pronounced septal bumps.

Referring still to FIG. 1, in the deployed or expanded condition of the frame 110, the bottom struts of the enlarged transition cells 144, to which the CAFs 150 are connected, extend in the outflow direction substantially parallel to the central longitudinal axis of the prosthetic heart valve 100. With this positioning, the CAFs 150 may be positioned in alignment with, or nearly in alignment with, the smallest diameter portion of the frame 110 at the central portion 140. In other words, the CAFs 150 of frame 110 are effectively cantilevered. This cantilevering of the CAFs 150, if no additional support is provided, may result in certain disadvantages. As explained above, the prosthetic leaflets 170 are coupled to the CAFs 150. As a result, during ventricular systole when the prosthetic leaflets 170 are closed and pressure is applied in the ventricular-to-atrial direction, the CAFs 150 and the struts of the enlarged transition cells 144 to which the CAFs 150 are attached may deflect radially inwardly toward each other. Although some amount of deflection may be desirable, the length of the CAFs 150 may be such that a risk of over-deflection may result. If the CAFs 150 deflect too much during ventricular systole, the prosthetic leaflets 170 may not coapt correctly, leading to inefficient valve functionality. Also, another disadvantage of large amounts of deflection of the CAFs 150 is that the struts from which the CAFs 150 extend may fatigue rapidly, possibly leading to failure of the frame 110.

Other potential disadvantages may result if the CAFs 150 of frame 110 do not have additional support. For example, as the prosthetic heart valve 100 is deployed from a delivery device, the ventricular or outflow end of the frame may exit the delivery device first, and begin to self-expand while the atrial end or inflow end of the frame remains collapsed within the delivery device. While the atrial portion 120 remains within the delivery device, a lever type of effect may result in which the CAFs 150 tend to splay radially outwardly as the prosthetic heart valve 100 begins to deploy from the delivery device. If the CAFs 150 are not separately supported, the CAFs 150 may tend to splay to a position that is radially outward of the shape-set position. As a result of this splaying, the prosthetic leaflets 170 may be pulled or stretched. Even if this splaying occurs temporarily during delivery, the prosthetic leaflets 170 (and/or the sutures connecting the prosthetic leaflets 170 to the CAFs 150) may be damaged, stressed, or otherwise weakened enough to cause a risk that the prosthetic leaflets 170 may either not function correctly upon implantation, or even if the prosthetic leaflets 170 function appropriately upon implantation, the longevity of the prosthetic leaflets 170 may be reduced as a result of the stress during splaying of the CAFs 150.

A third potential disadvantage may result if the CAFs 150 of frame 110 do not have additional support. Because the CAFs 150 are connected to the ventricular portion 130 of the frame 110, deformation of the ventricular portion 130 of the frame may result in deformation of the CAFs 150, and particularly their positions relative to each other. When the ventricular portion 130 deforms, the CAFs 150 may deform out of their generally circular or cylindrical alignment. This may be undesirable because as the CAFs 150 deform away from their shape-set, generally circular or cylindrical alignment, the prosthetic leaflets 170 become less likely to properly coapt with each other to form a seal.

In order to address any one or more of the potential disadvantages of CAFs 150 that exclude additional support members, a commissure support member 180 (which may be referred to herein as a CAF support or simply a support member) may be provided. The CAF support 180 is shown assembled to the frame 110 in FIG. 1. The CAF support 180 may take various forms, but in some examples it may be an expandable and collapsible ring-shaped structure.

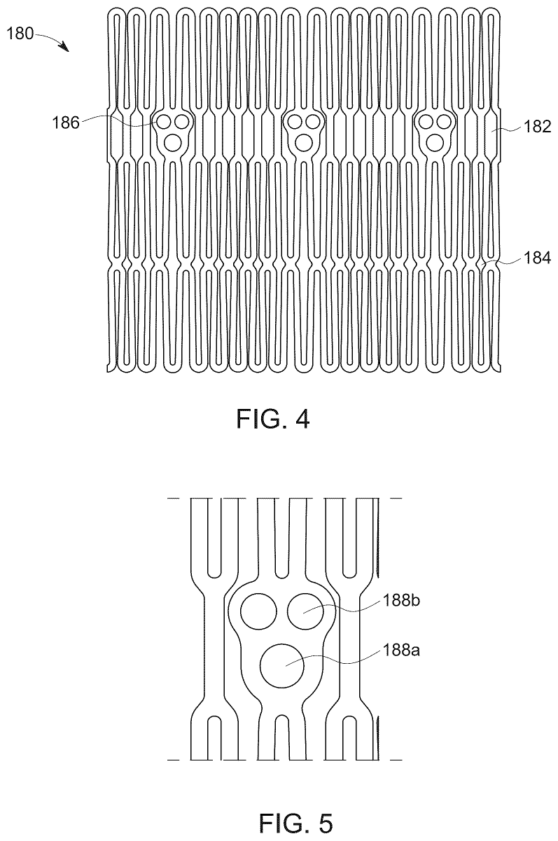

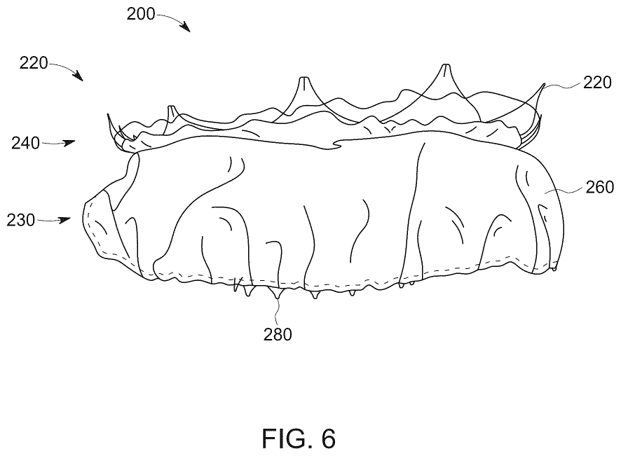

FIG. 4 shows a cut pattern for one example of CAF support 180. In the embodiment of FIG. 4, the CAF support 180 is formed of a shape-memory material, such as nitinol, and may be laser-cut from a nitinol tube using a pattern similar to that shown in FIG. 4. In the illustrated embodiment, commissure support 180 includes a first row of cells 182 and a second row of cells 184, and integrated connectors 186 provided on the commissure support 180. As shown in the enlarged view of FIG. 5, the connector 186 may have a partial “dog-bone” shape that includes a central eyelet 188a and two eyelets 188b arranged in a horizontal pair adjacent to the central eyelet 188a. In this particular embodiment, the three eyelets of the connector 186 may provide for a three-point connection to the corresponding CAF 150. In particular, the two horizontally arranged eyelets 188b may align with either pair of eyelets 154, with the central eyelet 188a aligning with an eyelet 152 in the column of eyelets of the CAF 150 positioned adjacent to the relevant pair of horizontal eyelets 154.

The symmetry of the eyelets in the CAF 150 may allow for the commissure support 180 to be coupled to the frame 110 in two different orientations. For example, the commissure support 180 may be coupled to frame 110 with the connectors 186 aligned with the inflow side of the CAFs 150, whereas in other embodiments, the opposite orientation may be used, in which the connectors 186 are aligned with the outflow side of the CAFs 150. In either orientation, the commissure support 180 may generally overlie the same portions of the frame 110. In other words, either orientation of the commissure support 180 may be used relative to the frame 110 without any significant deviation in the resulting functionality, but it may be desirable to have different options for assembly.

Although not shown in FIG. 1, a buffer material may be provided between the contact points of the commissure ring 180 and the frame 110, so that there is no or minimal direct metal-to-metal contact. Any buffer material may be suitable, including fabric materials or tissue materials, and similar buffer materials may be provided with other embodiments described herein to prevent or minimize metal-to-metal contact between a frame and a commissure support.

After using the cut pattern of FIG. 4 on a tube of nitinol (or other material), the resulting structure of commissure support 180 may be shape set (e.g., via heat treatment) so that, in the absence of applied forces, the CAF support 180 forms a generally circular or cylindrical ring. In the expanded or unbiased condition, the interior diameter of CAF support 180 is about equal to the diameter of a circle that is aligned with the outer surfaces of the CAFs 150 when the frame 110 is in its expanded or unbiased condition.

The CAF support 180 may be positioned on the exterior of the CAFs 150 (and/or the cell struts from which the CAFs 150 extend) and coupled to the frame 110 via any suitable mechanism. For example, in some embodiments, the CAF support 180 may be sutured to the CAFs 150 and/or to the cell struts from which the CAFs 150 extend. Although suturing is described as one mechanism of fastening the CAF support 180 to the frame 110, it should be understood that other methods, such as adhesives, rivets (or other mechanical fasteners), etc. may be similarly suitable.

Commissure support 180 is shown and described above as being shape-set or otherwise configured into a generally circular or cylindrical shape, which would generally match the shape of the perimeter of the CAFs 150 when the prosthetic heart valve 100 is expanded and/or deployed. However, in some cases, the prosthetic leaflets 170 may open (e.g., during atrial systole) to an extent that would tend to extend radially outward of a circular perimeter formed along the CAFs 150. In other words, if the commissure support 180 was formed as a circle and coupled to the outer surfaces of the CAFs 150, the prosthetic leaflets 170 might be at risk of contacting the inner surface of the commissure support 180 when the prosthetic leaflets 170 open. This type of contact would generally be undesirable. In order to mitigate this concern, commissure support 180 may be shaped to provide clearance for the prosthetic leaflets 170 when they open, for example shaping the commissure support as a general ring structure with three enlarged lobes between each pair of connectors 186.

In use, even if the ventricular portion 130 of the frame 110 undergoes a significant amount of ovalization from forces acting on the frame 110, the commissure support 180 (and thus the CAFs 150) may maintain an almost perfect circular profile. Further, in use, the commissure support 180 may prevent the CAFs 150 from deflecting inwardly during ventricular systole more than desired, while also preventing the CAFs 150 from splaying outwardly more than desired during deployment of the prosthetic heart valve 100.

As should be understood from the disclosure provided herein, in some embodiments, a prosthetic heart valve includes a single frame (e.g., a nitinol frame) with a commissure support member to facilitate the prosthetic heart valve having a minimal profile with a wide treatment range of annular anatomy. The frame design and/or the commissure support member help to minimize the pressure of which the prosthetic heart valve exerts against the native valve annulus. The single layer frame helps to enable the prosthetic heart valve to be compressed inside of a catheter with a small diameter (e.g., <33 French or <30 French), with sealing achieved in part by a sealing fabric. The commissure support may help to ensure the long-term durability of the prosthetic leaflets and the ventricular portion of the frame. And while the disclosure provided herein may be applied to prosthetic heart valves for replacing mitral or tricuspid valves, these features may work particularly well with the tricuspid valve due to the lower ventricular pressures involved, which may reduce the need for a bulkier two-piece frame design. In addition, the native tricuspid valve does not have the more pronounced fibrous structure found in the native mitral valve. Thus, instead of utilizing the native structure surrounding the annulus (as is often done for a prosthetic mitral valve) that works well to handle the compression other prosthetic heart valves use, the prosthetic heart valve described herein may anchor within the tricuspid valve annulus via one or more of (i) light pressure or draping of the generally bell-shaped ventricular portion of the frame; (ii) ventricular tines providing frictional engagement with the native valve annulus; and/or (iii) parachuting of the sealing skirt assisting with fixation.

It should be understood that, although the prosthetic heart valve 100 is described as including a frame 110 and a separate commissure support 180, the inclusion of the commissure support 180 does not significantly increase the profile of the prosthetic heart valve 100 when in the collapsed condition, compared to more traditional two-framed valves that may be used in mitral valve prostheses. Additional features of, and alternate embodiments of, prosthetic heart valve 100 are described in greater detail in U.S. Patent Application Publication No. 2024/0164895, the disclosure of which is hereby incorporated by reference herein. Features of prosthetic tricuspid valves generally are described in greater detail in U.S. Patent Application Publication No. 2023/0277304, the disclosure of which is hereby incorporated by reference herein.

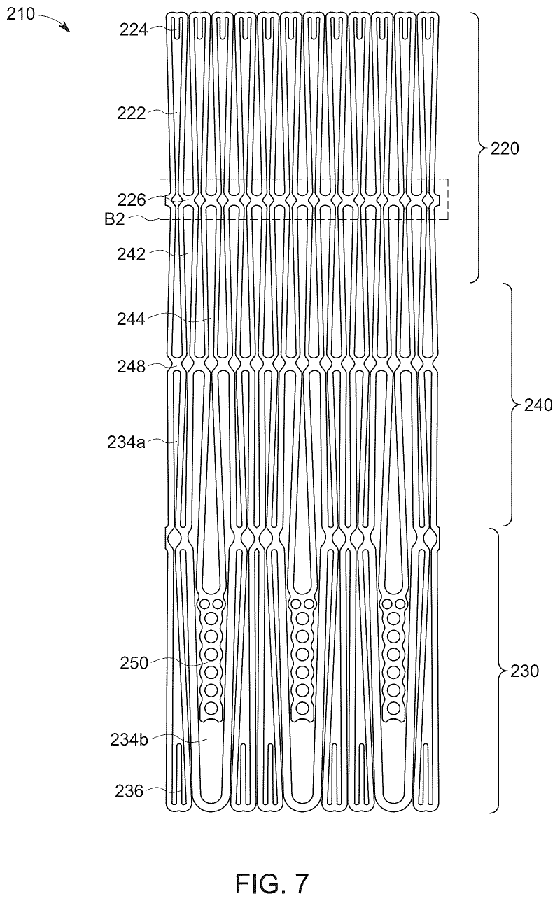

FIG. 6 illustrates a prosthetic heart valve 200 that is generally similar to prosthetic heart valve 100, but which has a significantly shorter axial length in the collapsed condition. Generally, prosthetic heart valve 200 may include a frame 210. Like frame 110, frame 210 may include an atrial anchor or disk 220, a ventricular anchor or disk 230, and a central or waist portion 240. Frame 210 may be similar or identical to frame 110 in most aspects, with a major differentiator being that frame 210 includes a waist portion 240 with a larger diameter, which helps results in a smaller crimped length, as described in greater detail below. Prosthetic heart valve 200 may include a commissure support 280 (only a portion being visible in FIG. 6) that is similar or identical to commissure support 180, and which is not described in more detail again. Prosthetic heart valve 200 may include a plurality of prosthetic leaflets 270 and a sealing skirt 260, which may both be similar or identical to prosthetic leaflets 170 and skirt 160, except for any specific differences described below.

There are significant potential benefits to a prosthetic atrioventricular valve that has a shorter length (in the inflow-to-outflow direction) while collapsed in a delivery device. For example, such a shorter valve will be more deployable in a larger range of patients compared to longer valves. On average, the distance between a patient's tricuspid valve annulus and a position of the wall of the right ventricle aligned with the annulus is about 42 mm. However, this distance is around 20 mm for about 5% of patients. In other words, when a prosthetic atrioventricular valve is in the collapsed condition within a delivery device, it may be too long to be able to be positioned in the desired location relative to the native valve annulus before the leading/distal end of the delivery device contacts the wall of the ventricle, making it difficult or impossible to deploy the prosthetic valve in the desired configuration relative to the native valve annulus.

Prosthetic heart valve 100 may be referred to herein as the “small waist” valve design. Referring back to the small waist valve design, prosthetic heart valve 100 may have a minimum diameter of about 29 mm in the expanded condition, which may be the diameter of the frame 110 at the point of inflection 148. This is about the same as the diameter of the valve assembly (e.g. the assembled prosthetic leaflets 170) at the inflow end. The largest expanded diameter of the atrial and/or ventricular anchors 120, 130 required to treat native atrioventricular valves (particularly the tricuspid valve) is about 75 mm. The arc length of the expanded prosthetic heart valve frame, and resulting laser cut frame height, is generally determined by these two diameters. One way to shorten the crimped length of the prosthetic heart valve is to increase the expanded diameter of the waist portion of the frame. For example, for each unit of distance that the expanded diameter of the waist portion is increased, the crimped length of the prosthetic heart valve may be reduced by about 1.25 units of distance.

The “small waist” embodiment of prosthetic heart valve 100, and particularly frame 110, described in connection with FIGS. 1-2, may have an axial height or length of about 67 mm when collapsed within a delivery device. However, prosthetic heart valve 200 (which may be referred to as the “large waist” embodiment) includes frame 210, described in greater detail below, which may have a significantly smaller crimped height or length of about 53 mm (although other values, including between about 48 mm and about 58 mm may be suitable). This decrease of about 14 mm from about 67 mm (in the small-waisted embodiment) to 53 mm (in the large-waisted embodiment) may be achieved by increasing the expanded diameter of the waist portion 240 to about 40 mm, compared to the expanded diameter of waist portion 140 being about 29 mm (in the small-waisted embodiment). Thus by increasing the diameter of the waist portion by about 11 mm, the height of the frame may be decreased by about 14 mm (a ratio of about 1:1.25). Although this increased waist diameter of about 40 mm is one example to decrease the length of the crimped valve, it should be understood that other specific amounts of waist-diameter increase may be used for correspondingly different reductions in crimped valve length. Such shorter lengths, as described above, may allow for more patients to be treated, even those with relatively small clearance spaces available within the chamber of heart. In some embodiments, the frame 210 may come in various pre-set sizes, for example with expanded ventricular disk 230 having a diameter of about 50 mm, about 55 mm, about 60 mm, about 65 mm, about 70 mm, or about 75 mm. In some embodiments, the expanded waist diameter may be about 29 mm if the ventricular disk 230 size is about 50 mm or about 55 mm, about 32 mm if the ventricular disk 230 size is about 60 mm, about 35 mm if the ventricular disk 230 size is about 65 mm, about 40 mm if the ventricular disk 230 size is about 70 mm, or about 45 mm if the ventricular disk 230 size is about 75 mm.

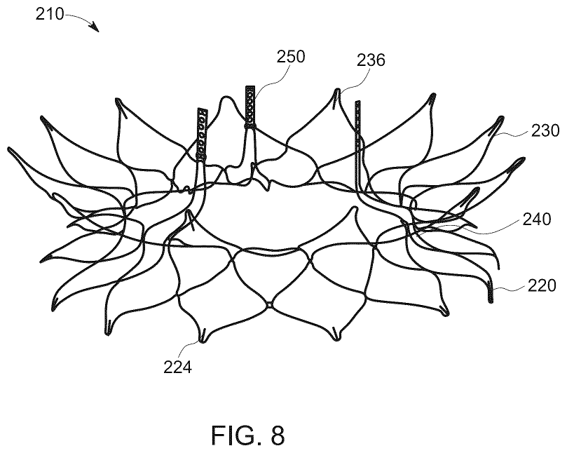

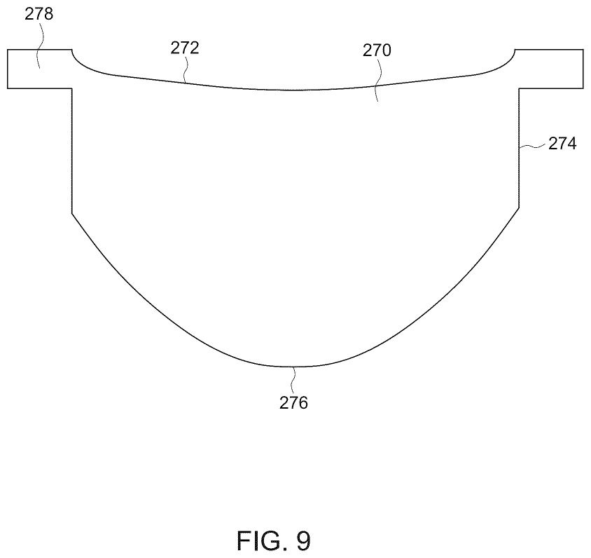

FIG. 7 illustrates a cut pattern of the frame 210 of prosthetic heart valve 200, as if cut longitudinally and laid on a table. It should be understood that the cut pattern of frame 110, shown in FIG. 2, is not provided to scale with the cut pattern of frame 210, shown in FIG. 7. FIG. 8 illustrates the frame 210, isolated from other components of the prosthetic heart valve 200, in the expanded condition. However, it should be understood that the orientation of the frame 210 in FIG. 8 is the opposite of that shown in FIG. 7. In other words, the top of the view of FIG. 7 corresponds to the inflow end, while the top of the view of FIG. 8 corresponds to the outflow end.

Referring to FIGS. 7-8, it should be understood that many features of frame 210 may be similar or identical to frame 110. For example, the materials and processes used to form the frame 210, other than the specific cut pattern and the specific shape to which the frames are set, may be the same, and thus are not all described in full detail again here. For example, frame 210 may include an atrial anchor or disk 220, a ventricular anchor or disk 230, and a central waist 240. Frame 210 may include CAFs 250 that are similar or identical to CAFs 150, although the struts to which the CAFs 250 connect may be slightly different compared to the corresponding struts in frame 210.

The inflow end of frame 210 may include a row of generally diamond-shaped cells 222, which may include pins 224 similar to pins 124. As with atrial cells 122, atrial cells 222 may have an outflow end that forms an inflection point 248 where the outer diameter of the waist 240 is smallest. However, one difference between atrial cells 122 and atrial cells 222 is that, while atrial cells 112 include sides defined by elongated beams 126, the sides 226 of atrial cells 222 include no such beams (or otherwise include beams with much smaller height than beams 126). This modification leads to shortening of the atrial anchor 220, which can be readily seen by comparing the height of box B1 in FIG. 2 to the height of box B2 in FIG. 7.

As with frame 110, frame 210 may include a plurality of generally diamond-shaped transition cells 242 in a row that is adjacent to the atrial cells 222. Transition cells 242 may include an inflow portion on the inflow side of waist 240 and an outflow portion on the outflow side of waist 240. In some examples, the transition cells 242 may be axially centered about the inflection point 248. Also similar to frame 110, the row of transition cells 242 may include three enlarged transition cells 244 that terminate in CAFs 250. The structure and function of CAFs 250 may be similar or identical to CAFs 150. Although CAFs 250 are shown with only one horizontal row of apertures, CAFs 250 may be modified to be identical to CAFs 150, or vice versa.

Although CAFs 250 may be similar or identical to CAFs 150, the struts of enlarged transition cells 244 that connect to CAFs 250 may be substantially longer than the corresponding struts of enlarged transition cells 144 that connect to CAFs 150. Referring to FIG. 8, although the diameter of waist 240 has been increased (to about 40 mm in the illustrated embodiment), it is still desirable for the CAFs 250 to be positioned along a circle that is approximately 29 mm in diameter (although the specific diameter may be modified). In other words, although frames 110 and 210 have significantly different diameters at their respective waists, the positions of the CAFs 150 compared to CAFs 250 are not significantly different. For example, when the prosthetic heart valve that incorporates frame 210 is in the expanded condition, the CAFs 250 may be positioned along an imaginary circle that has a diameter which is smaller than the diameter of waist 240. In order to achieve this, instead of having CAFs 150 that extend mostly straight downwardly from inflection points 148, the struts of enlarged transition cells 244 are contoured to with two curves that allow the CAFs 250 to be positioned a distance radially inward of the points of inflection 248. In order to allow for such curvatures, the struts connecting to CAFs 250 may need to be longer than the corresponding struts connecting to CAFs 150.

Referring again to FIG. 7, the ventricular anchor 230 of frame 210 may include a group of generally diamond-shaped first ventricular cells 234a, the inflow apex of which is an inflection point 248, and the outflow apex of which is an outflow end of the ventricular anchor 230. These cells may include tines 236 similar or identical to tines 136. The ventricular anchor 230 may include a group of second ventricular cells 234b that are generally similar to third ventricular cells 134c. The second ventricular cells 234b may be positioned between certain pairs of first ventricular cells 234a, and may include struts that extend from the inflection point 248 to the terminal outflow end of the ventricular portion 230. With this configuration, at least in the cut pattern shown in FIG. 7, the CAFs 250 may be thought of as either nested within second ventricular cells 234b or forming a boundary of second ventricular cells 234b. As should be understood from the description above, the modifications made to frame 210, compared to frame 110, allow the crimped height of the frame 210 to be significantly reduced by increasing the diameter of the waist portion 240, while still allowing the CAFs 250 to be positioned along a circle of about 29 mm. This, in turn, may allow prosthetic heart valve 200 to treat a wider selection of patients for the reasons described above.

A number of other features described in connection with prosthetic heart valve 100 are not described again in connection with prosthetic heart valve 200 because the features may be similar or identical. For example, outer skirt 260 may be substantially identical to outer skirt 160, including the sealing functionality, the outer skirt 260 being coupled (e.g., via sutures) to the waist portion 240 of the frame 210, the use and structure of the commissure support ring 280, the structure of the commissures 250 as cantilevered or floating commissures, the use of friction members such as tines 236, the use of atrial pins 234, and the way in which the atrial section of the outer skirt 260 is coupled to the frame 210.

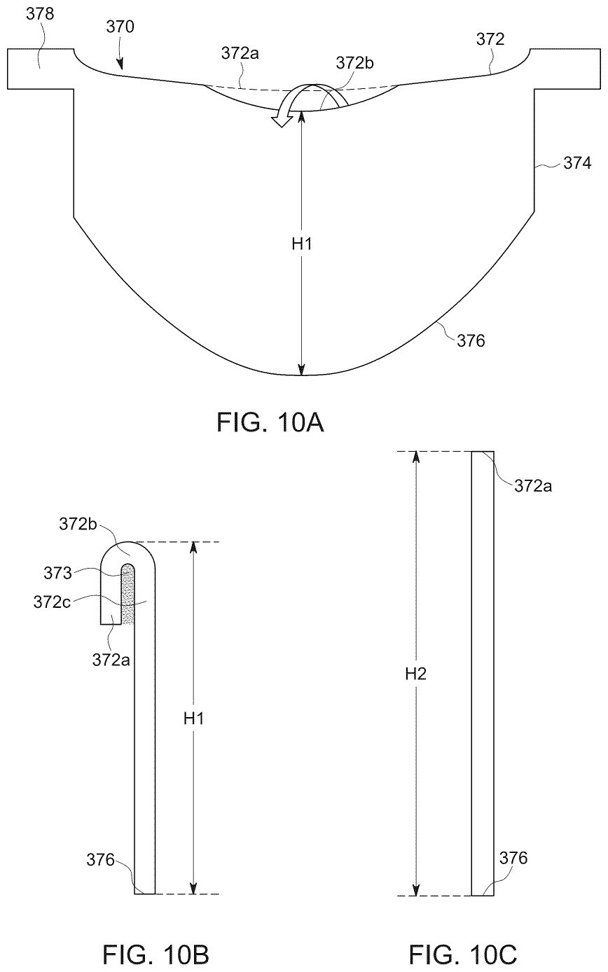

As with prosthetic leaflets 170, prosthetic leaflets 270 may be formed of any suitable tissue (e.g. pericardial tissue) or synthetic material (e.g. PET, PTFE, or UHMWPE). FIG. 9 illustrates an example of a prosthetic leaflet 270, as if laid flat on a table prior to being assembled with other components of prosthetic heart valve 200. Prosthetic leaflet 270 may include a free edge 272 at an outflow aspect of the prosthetic leaflet 270, the free edge 272 configured to move toward and away the free edges of the other prosthetic leaflets to create the valve functionality. Prosthetic leaflet 270 may include two side edges 274, with the side edge 274 of one prosthetic leaflet 270 configured to connect to a side edge 274 of an adjacent prosthetic leaflet 270, with the two side edges 274 being coupled together and to CAF 250. The prosthetic leaflet 270 may also include an attached edge 276 opposite the free edge 272 and extending between the side edges 274. The attached edge 276 may have a curved shape, including the general shape of a parabola or catenary. In some examples, the prosthetic leaflets 270 may include tabs 278, each tab 278 connecting an end of the free edge 272 to a corresponding end of the attached edge 276. If tabs 278 are included, a tab 278 of one prosthetic leaflet 270 may be coupled to a tab 278 of an adjacent leaflet 270, with the coupled tabs being coupled to the frame 210 via a CAF 250.

It is important for the attached edges 276 of each of the prosthetic leaflets 270 to be coupled to other structures of the prosthetic heart valve 200 in order to ensure a proper seal when the free edges 272 of the prosthetic leaflets 270 are coapted with each other. Referring briefly back to FIG. 1, the attached edges of the prosthetic leaflets 170 may be sutured to seal 160 (and/or a separate fabric) at or near the waist portion 140 to help ensure that blood can only flow through the center of the assembly of prosthetic leaflets 170. This may be relatively straightforward since, for frame 110, the diameter of the waist 140 is about the same as the diameter of a circle positioned over the CAFs 150. In other words, in prosthetic heart valve 100, the attached edges of the prosthetic leaflets 170 are positioned close to other structure of the prosthetic heart valve 100 that the attached edges of the prosthetic leaflets 170 may be coupled to (e.g. via suturing). For the large-waisted prosthetic heart valve 200, on the other hand, there may be a significant gap between the attached edges 276 of the prosthetic leaflets 270 and other structures of the prosthetic heart valve 200 (other than CAFs 250) that the attached edges 276 can be coupled to. This is because, at least in part, of the larger diameter waist 240 and resulting contoured struts that attach to CAFs 250. Put simply, the attached edges 276 of the prosthetic leaflets 270 cannot simply be sutured to the waist 240 and/or sealing fabric 260 at the waist 240.

To solve the problem addressed in the paragraph above, an additional leaflet securing material may be provided. The additional leaflet securing material may be nonpermeable to blood to help create the desired seal with the attached edges 276. Examples of such additional leaflet securing materials are described in greater detail in U.S. patent application Ser. No. 18/778,173, filed Jul. 19, 2024, the disclosure of which is hereby incorporated by reference herein.

In an exemplary use of the prosthetic heart valves described herein, a prosthetic heart valve may begin in the expanded condition prior to implantation into a patient. As described above, the prosthetic heart valve may include a single monolithic or unitary stent with an outer fabric on the stent, with a commissure support member (either circular or lobed) circumscribing the commissure attachment features. The prosthetic heart valve may be drawn or otherwise forced into a delivery catheter, the prosthetic heart valve transitioning into the collapsed condition as it moves into the delivery catheter. The outer diameter of the catheter of the delivery device may have a size of 30 French (10 mm) or smaller, including 28 French (9.33 mm) or smaller or 24 French (8 mm) or smaller. With the prosthetic heart valve successfully collapsed in the small diameter delivery device catheter, the delivery device may be introduced into the patient, for example through the femoral vein, and navigated to the target site, for example the native tricuspid valve. Upon reaching the target site, the prosthetic heart valve may be deployed from the delivery device catheter, for example by retracting the delivery device catheter relative to the prosthetic heart valve. As the constraint on the prosthetic heart valve is removed, the prosthetic heart valve will naturally begin to expand as the frame tends to return to its preset shape. In some examples, the ventricular disk of the prosthetic heart valve is released first within the ventricle (e.g., the right ventricle). As the ventricular disk expands, the ventricular disk may begin to apply light pressure on the tissue, and the ventricular tines may frictionally engage (with or without piercing) the native tissue. As deployment continues, the center portion of the stent of the prosthetic heart valve will generally align with the valve annulus. As the atrial side of the prosthetic heart valve deploys, the atrial disk of the stent will expand on the atrial side of the native valve. Thus, as described above, a small delivery device may be used, despite the requirement of coverage of a large native valve annulus area, and without losing any sealing capabilities despite using only a single stent with a small center portion housing the prosthetic leaflets.

Various designs of prosthetic leaflets and/or prosthetic heart valves are described below, at least some of which may include features to allow for intentional regurgitation, and in some examples intentional regurgitation that decreases over time. Although such prosthetic leaflets may have particular use in a prosthetic tricuspid valve, including prosthetic heart valve 100 and/or prosthetic heart valve 200 described above, it should be understood that any of the prosthetic leaflets described below may be incorporated into any prosthetic heart valve (including prosthetic aortic, pulmonary, tricuspid, or mitral valves), whether having designs similar to prosthetic heart valve 100 or prosthetic heart valve 200 or having designs different from prosthetic heart valve 100 or prosthetic heart valve 200, as long as intentional regurgitation (and/or changing leaflet shape, dimensions, or maximum effective axial height/distance or maximum effective length/width over time) is desired. Thus, the specifics of the non-leaflet portions of a prosthetic heart valve, including those described above, should not be considered to limit the applicability of the prosthetic leaflets described in greater detail below.

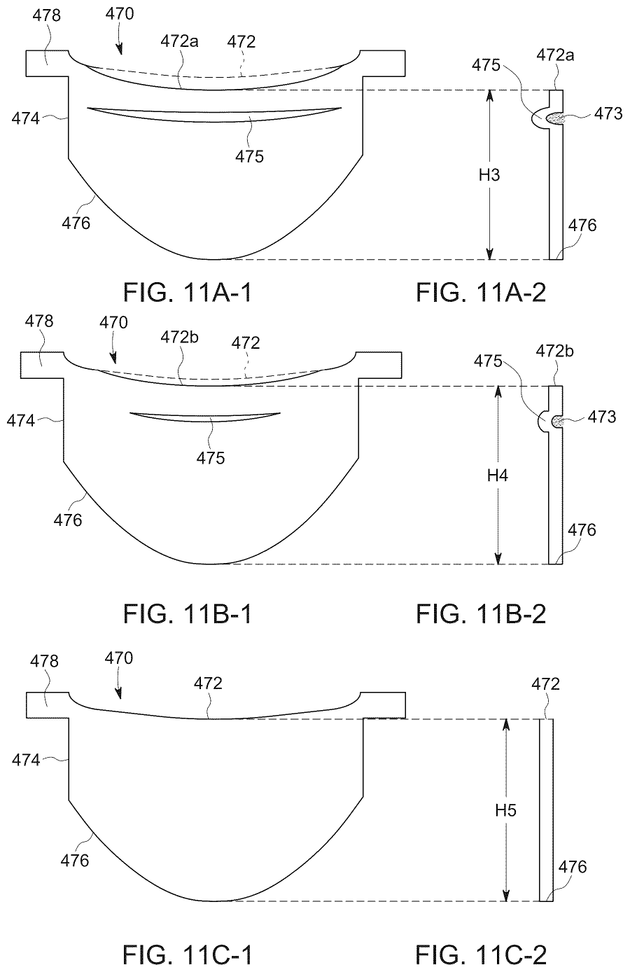

Referring now in addition to FIG. 10A, FIG. 10A is a front view of a prosthetic leaflet 370, as if laid out on a table, according to an aspect of the disclosure. Prosthetic leaflet 370 may be similar or identical to prosthetic leaflet 270, at least in some conditions. For example, prosthetic leaflet 370 may be formed of any of the materials described in connection with prosthetic leaflet 270, and three prosthetic leaflets 370 (or two or four or more of the prosthetic leaflets) may be used to create a valve-type function with any prosthetic heart valve, including for example prosthetic heart valve 100 or prosthetic heart valve 200. The prosthetic leaflet 370 may include an attached edge 376, which may be similar or identical to attached edge 276, which may be sutured or otherwise coupled to a component of a prosthetic heart valve, such as an inner skirt and/or frame. In some examples, prosthetic leaflet 370 may include side edges 374 and/or tabs 378, which—if included—may be substantially similar or identical to the side edges 274 and tabs 278 of prosthetic leaflet 270.

FIG. 10A illustrates the free edge 372 of the prosthetic leaflet 370 may have a terminal edge portion 372a shown in dashed lines that, prior to assembly of the prosthetic heart valve (which may be referred to as a pre-use condition), follows the same or similar contour as the free edge 272 of prosthetic leaflet 270. However, the terminal edge portion 372a of the free edge 372 may be folded over so to form a modified free edge portion 372b. Upon being folded over, a surface of the folded terminal edge portion 372a may be fixed to a confronting surface of a remaining portion 372c of the prosthetic leaflet 370, for example as shown in FIG. 10B. When the prosthetic leaflet 370 is assembled as a component of a prosthetic heart valve, the folded terminal edge portion 372a may be positioned either on the luminal surface of the prosthetic leaflet 370 (e.g. the portion generally facing the radial interior of the prosthetic heart valve) or on the abluminal surface of the prosthetic leaflet 370 (e.g. the portion generally facing the radial exterior of the prosthetic heart valve).

Referring now in addition to FIG. 10B, in some embodiments, the fixation of the terminal free edge portion 372a to the remaining portion 372c may be a permanent connection so that the shape of the modified free edge portion 372b is permanent. Suitable permanent fixation mechanisms include, for example, non-degrading (or substantially non-degrading) adhesives, binding-type mechanisms, agents, or materials, or other suitable fixation mechanisms capable of providing an equivalent function. In the example illustrated in FIG. 10B, a temporary fixation mechanism 373 is used to fix the terminal free edge portion 372a to the remaining portion 372c. In the example shown, the temporary fixation mechanism 373 is a biocompatible adhesive that transitions (e.g. dissolves, degrades, absorbs, or releases) over time, although other biocompatible binding-type mechanisms, agents, or materials that transition (e.g. dissolve, degrade, absorb, or release) over time may be suitable.

As best shown in FIG. 10B, while the terminal free edge portion 372a is fixed to the remaining portion 372c, the modified free edge portion 372b acts as a functional free edge (with the understanding that the structure is not necessarily an actual or technical “edge”) of the prosthetic leaflet 370 that will move toward the free edge(s) (or functional free edge(s)) of the other prosthetic leaflet(s) that form the valve assembly of the prosthetic heart valve when blood pressure tends to force the prosthetic leaflets into a coapted or closed condition. While the terminal free edge portion 372a is fixed to the remaining portion 372c, the modified free edge portion 372b extends an effective axial height/distance H1, which may be the maximum effective axial height/distance of the prosthetic leaflet 370 in this condition. This effective axial height/distance H1 is too small to allow for sufficient coaptation with the free edges (or functional free edges) of other prosthetic leaflets to fully prevent backflow through the valve assembly when the prosthetic leaflets are in the closed condition. It should be understood that, although the term “closed condition” is used, such terminology does not require total (i.e. 100%) prevention of regurgitation through the valve assembly despite the leaflets being in a closed condition. While the modified free edge portion 372b of prosthetic leaflet 370 has the axial height/distance H1, the prosthetic heart valve that incorporates prosthetic leaflet 370 allows for intentional regurgitation. As described above, this intentional regurgitation may be useful in certain situations, including for example in prosthetic tricuspid valve replacements in which risk to the patient may be created if tricuspid regurgitation is resolved too much and/or too quickly.

As noted above, in some examples, the use condition shown in FIG. 10B may be a permanent use condition if the fixation mechanism 373 is permanent. Although this permanent use condition may be better suited for tricuspid valve replacements for which some amount of regurgitation may be well tolerated, it should be understood that the permanent use condition shown in FIG. 10B may be used for a prosthetic leaflet 370 in any heart valve replacement if desired.

In other examples, the use condition shown in FIG. 10B may be a temporary or initial use condition. In this example, the initial use condition may create intentional regurgitation in the prosthetic heart valve that incorporates prosthetic leaflet 370, but only for a limited amount of time. For example if the fixation mechanism 373 is a temporary fixation mechanism such as a dissolvable or degradable adhesive or other bonding-type mechanism, agent, or material, as the fixation mechanism 373 transitions over time (e.g., due to the environment in situ), the modified free edge portion 372b will eventually unfold or unfurl as degradation/dissolving occurs so that, as shown for example in FIG. 10C, the terminal edge portion 372a becomes the functional free edge. In this final use condition, which may be similar or identical to the use condition of prosthetic heart valve 270, the terminal edge portion 372a extends an effective axial height/distance H2 (which may be the maximum effective axial height/distance of the prosthetic leaflet 370 in this condition), which is greater than the effective axial height/distance H1. This effective axial height/distance H2 is sufficiently large to allow for sufficient coaptation with the free edges (or functional free edges) of other prosthetic leaflets to fully prevent backflow or to substantially fully prevent backflow (e.g. between about 0 mL and about 5 mL of regurgitation per beat) through the valve assembly when the prosthetic leaflets are in the closed condition. Stated in other words, the effective axial height/distance H1 of the functional free edge of the initial use condition (in which the functional free edge of the prosthetic leaflet 370 is the modified free edge portion 372b) is smaller/less than the effective axial height/distance H2 of the functional free edge of the final use condition (in which the functional free edge is the terminal edge portion 372a of the prosthetic leaflet 370). It should also be understood that the shape of the prosthetic leaflet 370 in the final use condition illustrated in FIG. 10C may be substantially similar or identical to the shape of the prosthetic leaflet 370 in the pre-use condition described in connection with FIG. 10A (e.g. prior to folding the edge of the leaflet).

In some examples, the fixation mechanism 373 may include or be formed of (i) fibrin glue, (ii) polysaccharide, polypeptide, or polymeric adhesives, (iii) nonbiodegradable polymers (e.g. poly(ethylene glycol)-based hydrogel adhesives) copolymerized with degradable polymers, (iv) biomimetic tissue adhesives (e.g., mussel, gecko, sandcastle worm or silk inspired adhesives), or (v) combinations or alternatives thereof. Further details of certain known adhesives are described in greater detail in Bhagat, Vrushali, and Matthew L. Becker, “Degradable adhesives for surgery and tissue engineering.” Biomacromolecules 18.10 (2017): 3009-3039. Adhesive elements may include binding elements including or incorporating elements that are not typically defined or marketed as “adhesives” but function to temporarily position or bind one or more leaflets in a deformed or restricted state and are configured to transition over time, to an undeformed (or less deformed) or unrestricted (or less restricted) state. In examples in which the fixation mechanism 373 is temporary, the particular fixation mechanism 373 may be selected in order to allow for regurgitation for a desired amount of time before the fixation mechanism 373 degrades or dissolves, including for example 1 week, 1 month, 6 months, 9 months, 1 year, etc. It should be understood that the time periods noted above are merely exemplary.

The prosthetic heart valve that incorporates prosthetic leaflet 370 may include only one prosthetic leaflet 370 with the modified free edge portion 372b (whether permanent or temporary), or more than one such prosthetic leaflet 370. For example, if a prosthetic heart valve includes three prosthetic leaflets, one, two, or all three of the prosthetic leaflets may have a modified free edge portion 372b similar or identical to that shown in and described in connection with FIGS. 10A-10C. Further, if more than one prosthetic leaflet 370 with a modified free edge portion 372b is provided, the modified free edge portions 372b may all be on the luminal surface of the corresponding prosthetic leaflet 370, may all be on the abluminal surface of the corresponding prosthetic leaflet 370, or one or more of the prosthetic leaflets 370 may include a modified free edge portion 372b on the abluminal surface while one or more of the remaining prosthetic leaflets 370 may include a modified free edge portion 372b on the luminal surface. For example, including a modified free edge portion 372b on the abluminal surface of the prosthetic leaflet 370 may create a larger amount of regurgitation than if the modified free edge portion 372b were included on the luminal surface. Thus, the number of prosthetic leaflets 370 that include the modified free edge portion 372b, as well as the luminal or abluminal positioning of the modified free edge portion 372b, may be tailored to create a larger or smaller amount of regurgitation as desired.

Although FIG. 10A shows the modified free edge portion 372b being positioned along a particular length that is smaller than the entire length of the free edge 372 and being positioned generally centered about the center of the free edge 372 (e.g. generally centered between the side edges 374), it should be understood that this positioning is merely exemplary. Thus, in other examples, the modified free edge portion 372b may have a smaller or larger length than shown, and may be positioned at different locations between the tabs 378 (and/or between the side edges 374) than is shown in FIG. 10A. Further, although FIG. 10A illustrates a single modified free edge portion 372b, in some examples, two or more modified free edge portions 372b may be positioned along the free edge 372 of the prosthetic leaflet 370. Still further, although FIGS. 10A-10B illustrate a particular exemplary effective height/distance H1 (e.g. a particular amount overlap between the folded portion of the terminal edge portion 372a and the remaining portion 372c), it should be understood that in other embodiments, the effective height/distance H1 and/or the amount of overlap may be increased or decreased compared to that shown, which may for example decrease or increase (respectively) the amount of regurgitation while the prosthetic leaflet 370 is in the initial use condition similar to that shown in FIG. 10B.

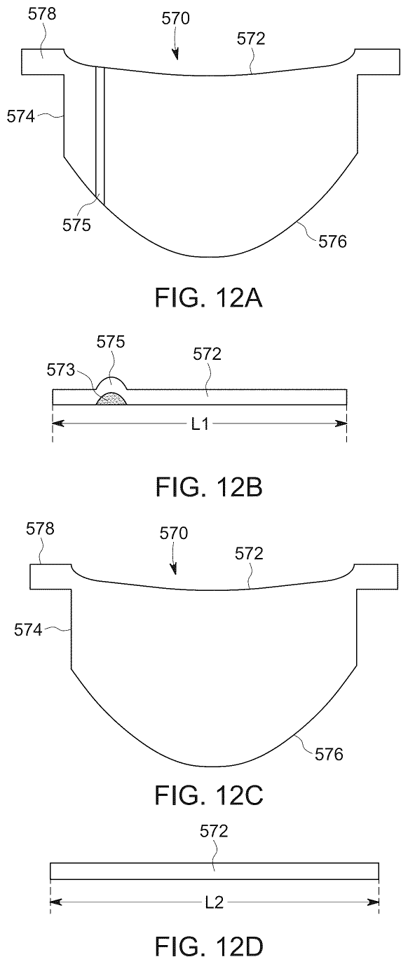

Referring now in addition to FIG. 11A-1, FIG. 11A-1 is a front view of a prosthetic leaflet 470, as if laid out on a table, according to an aspect of the disclosure. Prosthetic leaflet 470 may be similar or identical to prosthetic leaflet 270 and/or 370, at least in some conditions. For example, prosthetic leaflet 470 may be formed of any of the materials described in connection with prosthetic leaflet 270 or 370, and three prosthetic leaflets 470 (or two or four or more of the prosthetic leaflets) may be used to create a valve-type function with any prosthetic heart valve, including for example prosthetic heart valve 100 or prosthetic heart valve 200. The prosthetic leaflet 470 may include an attached edge 476, which may be similar or identical to attached edge 276 or 376, which may be sutured or otherwise coupled to a component of a prosthetic heart valve, such as an inner skirt and/or frame. In some examples, prosthetic leaflet 470 may include side edges 474 and/or tabs 478, which—if included—may be substantially similar or identical to the side edges 274, 374 and tabs 278, 378 of prosthetic leaflets 270, 370.

FIG. 11A-1 illustrates an exemplary position of the free edge 472 of the prosthetic leaflet 470 in dashed lines to show an unmodified or pre-use or pre-assembled condition. In the condition of FIG. 11A-1 and 11A-2, the prosthetic leaflet 470 may be modified so that the free edge 472 has a first modified free edge position 472a. For example, referring to FIGS. 11A-1 and 11A-2, a section of the prosthetic leaflet 470 may be pinched, folded, or otherwise gathered, to form a gathered section 475. For example, a length of material forming the prosthetic leaflet 470 may be gathered so that two portions of the same surface (e.g. either the surface that will face radially inwardly or radially outwardly upon formation of the prosthetic heart valve) contact or at least confront each other. The gathered section 475 may be fixed in the gathered configuration using a fixation mechanism 473. The fixation mechanism 473 may be any of those described above in connection with fixation mechanism 373, including for example a permanent or temporary fixation mechanism, and including for example an adhesive or other binding-type mechanism, agent, or material.

As shown in FIGS. 11A-1 and 11A-2, while gathered section 475 is maintained in the initial use condition, the natural or unmodified free edge 472 is maintained in a first modified free edge position 472a in which an effective axial height/distance H3 (e.g. in an axial direction of blood flow, and which may be the maximum effective axial height/distance of the prosthetic leaflet 470 in this condition) between the attached edge 476 and the free edge is smaller/less than the natural or unmodified effective axial height/distance H5 (shown and described in connection with FIGS. 11C-1 and 11C-2). Similar to the effect of effective axial height/distance H1 described in connection with FIGS. 10A-10B, the effective axial height/distance H3 is too small to allow for sufficient coaptation with the free edges of other prosthetic leaflets to fully prevent backflow through the valve assembly when the prosthetic leaflets are in the closed condition. While the free edge 472 of the prosthetic leaflet 470 has the first modified free edge position 472a (and the corresponding effective axial height/distance H3), the prosthetic heart valve that incorporates prosthetic leaflet 470 allows for intentional regurgitation. If the fixation mechanism 473 is a permanent (or “substantially permanent” which is intended to be defined herein as a configuration/composition that may have the capability to be permanent during the valve lifespan but which may wear away to some de minimis degree due to inevitable wear and tear) fixation mechanism, the regurgitation may be permanent (or substantially permanent), which, as described above, may be tolerated in some situations while avoiding potential harm from immediately resolving all regurgitation. If the fixation mechanism 473 is temporary, the fixation mechanism 473, over time in situ, transitions (e.g. dissolves, releases, absorbs, or releases) to allow for a change in the position of the free edge 472.

Referring now in addition to FIGS. 11B-1 and 11B-2, FIGS. 11B-1 and 11B-2 illustrate the prosthetic leaflet 470 in an intermediate use condition in which the fixation mechanism 473 has partially transitioned (e.g., degraded, released, absorbed, or dissolved) to a different condition as compared to the initial use condition shown in FIGS. 11A-1 and 11A-2. As best seen by comparing FIGS. 11B-1 and 11B-2 to their counterpart FIGS. 11A-1 and 11A-2, the gathered section 475 has begun to separate or unfurl as the fixation mechanism 473 has begun to transition (e.g. degrade or dissolve). As a result of the (at least partial) reversal of the prior gathering of the gathered section 475, the free edge 472 has shifted from the first modified free edge position 472a to a second modified free edge position 472b which is closer to the natural or unmodified position of the free edge 472, which is represented by a dashed line in FIG. 11B-1. This corresponds to an effective axial height/distance H4 between the attached edge 476 and the free edge (at the second modified free edge position 472b and which may be the maximum effective axial height/distance of the prosthetic leaflet 470 in this condition) which is greater than the effective axial height/distance H3, but still smaller/less than the natural or unmodified height/distance H5 (shown and described in connection with FIGS. 11C-1 and 11C-2). Thus, as the free edge moves closer to the natural or unmodified position of the free edge 472, the prosthetic leaflet 470 has better coaptation with the remaining prosthetic leaflets, although still allowing for some amount of regurgitation through the valve, which is smaller/less than the amount of regurgitation allowed when the prosthetic leaflet 470 is in the initial use condition.

Referring now in addition to FIGS. 11C-1 and 11C-2, FIGS. 11C-1 and 11C-2 illustrate the prosthetic leaflet 470 in a final use condition (which may be the similar to or the same as a pre-assembled or pre-use condition) in which the fixation mechanism 473 has completely or substantially completely transitioned (e.g. degraded, released, absorbed, or dissolved) as also discussed with respect to other examples. As best seen by comparing FIGS. 11C-1 and 11C-2 to their counterpart FIGS. 11A-1, 11B-1, 11A-2, and 11B-2, the gathered section 475 has fully or substantially fully separated or unfurled as the fixation mechanism 473 fully or substantially fully transitioned (e.g. degraded or dissolved), so that the gathered section 475 has largely or completely disassociated from the leaflet 470. This condition may also or alternatively be defined by the lack of fixation mechanism 473 present on the leaflet 470. As a result of this complete (or substantially complete) reversal of the prior gathering of the gathered section 475, the free edge 472 has shifted from the second modified free edge position 472b to the natural or unmodified position of the free edge 472. It should be understood that the free edge 472 shown in FIG. 11C-1 has the same position as the dashed lines in FIGS. 11A-1 and 11B-1 representing the unmodified position of the free edge 472, which is actually achieved in the final use condition of FIG. 11C-1. This positioning of the free edge 472 corresponds to the effective axial height/distance H5 (which may be the maximum effective axial height/distance of the prosthetic leaflet 470 in this condition) between the attached edge 476 and the free edge 472 (at the unmodified or natural position) being greater than the effective axial heights/distances H3 and H4. Thus, as the free edge 472 achieves the natural or unmodified position, as shown in FIGS. 11C-1 and 11C-2, the prosthetic leaflet 470 has even better coaptation with the remaining prosthetic leaflets (compared to the initial and intermediate use conditions), preventing all or substantially all regurgitation through the valve (e.g. between about 0 mL and about 5 mL of regurgitation per beat), although it should be understood that the final use condition may instead correspond to an intentionally designed, non-zero amount of regurgitation.

As noted above, in some examples, the fixation mechanism 473 may be configured and consciously selected to be permanent so that there is no significant change in the shape of the prosthetic leaflet 470 over time, and no intentional change in regurgitation over time of the prosthetic heart valve that incorporates the prosthetic leaflet 470. In such examples, the amount of material used to form the gathered section 475 may be selected to increase the effective axial height/distance (e.g. by gathering a relatively small amount of material of the prosthetic leaflet 470 before fixing the gathered section) to achieve a relatively small amount of regurgitation, or decrease the effective axial height/distance (e.g. by gathering a relatively large amount of material of the prosthetic leaflet 470 before fixing the gathered section) to achieve a relatively large amount of regurgitation.

Further, in examples in which the fixation mechanism 473 is configured to be temporarily modifying the leaflet, the transition (e.g. degradation, dissolution, absorption, release, etc.) of the fixation mechanism 473 may be gradual, occur stepwise or be binary. In other words, although three total use conditions are shown in FIGS. 11A-1 through 11C-2, the fixation mechanism 473 may transition over time at a constant rate (or a non-constant rate) so that the change in effective axial height/distance is gradual, allowing for progressively better coaptation and progressively smaller regurgitation until the regurgitation is completely (or substantially completely) resolved to a desired degree. Therefore, there may be a plurality of intermediate conditions defined by varying effective axial heights/distances of the respective leaflet between the initial use and final conditions. In other examples, the fixation mechanism 473 may have more of a discrete condition transition or a binary release. For example, most, substantially all, or all of the material forming the fixation mechanism 473 may be configured to transition (e.g. degrade, dissolve, absorb or release) at about the same time point such that, as soon as the transition occurs, the gathered section 475 may unfurl or release at once to achieve a relatively abrupt transition (e.g. from an initial use condition which may have a configuration similar to FIG. 11A-1 or to FIG. 11B-1) to the final condition similar to that shown in FIGS. 11C-1 and 11C-2.

In examples in which the fixation mechanism 473 is temporary, as with fixation mechanism 373 described above, the particular fixation mechanism 473 may be selected in order to allow for regurgitation for a desired amount of time before the fixation mechanism 473 transitions (e.g. degrades or dissolves), including for example 1 week, 1 month, 6 months, 9 months, 1 year, etc. It should be understood that the time periods noted above are merely exemplary and will ultimately be selected based on the desired rate for a particular application, the properties of available fixation mechanism materials and the individual conditions in situ affecting degradation/dissolution rates.

Further, the prosthetic heart valve that incorporates prosthetic leaflet 470 may include only one prosthetic leaflet 470 with a gathered section 475, or more than one such prosthetic leaflet 470. Although FIG. 11A-1 shows the gathered section 475 being initially positioned along a particular length that is smaller than the entire length of the free edge 472 and being positioned generally centered about between the side edges 474 and closer to the free edge 472 than to the attached edge 476, it should be understood that this positioning is merely exemplary. Thus, in other examples, the gathered section 475 may have a smaller or larger length than shown, and may be positioned at different locations (e.g. closer to either side edge 474 and/or closer to the free edge 472 or attached edge 476) than shown in the figures. Still further, it should be understood that, although prosthetic leaflet 470 is shown as having a single gathered section 475, a single prosthetic leaflet 470 may instead include two or more gathered sections 475. Further, it should be understood that a prosthetic leaflet may include both one or more gathered section 475 along with a modified free edge portion similar to that shown in FIGS. 10A-B. Further, a prosthetic heart valve that includes two or more leaflets may include one or more prosthetic leaflets having any of the configurations (including combinations) described above along with one or more additional prosthetic leaflets having the same configuration or any of the other configurations described above to fine-tune the amount of regurgitation, as well as the time profile by which the regurgitation reduces. As described in greater detail below, this optionality includes using different fixation mechanisms for each gathered portion and/or modified free edge with different time-release profiles, such that the individual gathered portions or modified free edges unfurl or release at different times.

While FIGS. 11A-1 and 11B-1 illustrate a prosthetic leaflet 470 with a gathered section 475 that runs generally horizontally (e.g. orthogonal to the direction of blood flow) to reduce the effective axial height/distance of the prosthetic leaflet 470 in order to reduce the amount of leaflet material available to contact other leaflets during coaptation, in other examples, a gathered section may run generally axially (e.g. along the direction of blood flow) to tension the leaflet, at least temporarily.

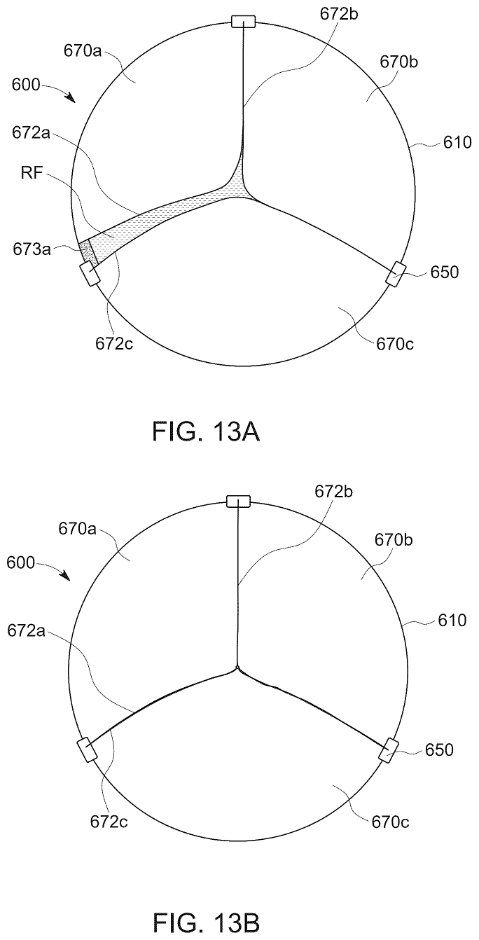

Referring now in addition to FIG. 12A, FIG. 12A is a front view of a prosthetic leaflet 570, as if laid out on a table, according to an aspect of the disclosure. Prosthetic leaflet 570 may be similar or identical to prosthetic leaflets 270, 370 and/or 470, at least in some conditions. For example, prosthetic leaflet 570 may be formed of any of the materials described in connection with prosthetic leaflet 270 or 370, and three prosthetic leaflets 570 (or two or four or more of the prosthetic leaflets) may be used to create a valve-type function with any prosthetic heart valve, including for example prosthetic heart valve 100 or prosthetic heart valve 200. The prosthetic leaflet 570 may include an attached edge 576, which may be similar or identical to attached edge 276 or 376, which may be sutured or otherwise coupled to a component of a prosthetic heart valve, such as an inner skirt and/or frame. In some examples, prosthetic leaflet 570 may include side edges 574 and/or tabs 578, which—if included—may be substantially similar or identical to the side edges 274, 374 and tabs 278, 378 of prosthetic leaflets 270, 370.

Referring now in addition to FIG. 12B, FIG. 12B is a top view (e.g. viewing the outflow or free edge 572 of the prosthetic leaflet 570 from above) of prosthetic leaflet 570. FIGS. 12A-12B both show prosthetic leaflet 570 in an initial use condition, which may be conceptually similar to the initial use condition of prosthetic leaflets 370 and 470. For example, in the condition of FIGS. 12A-12B, the prosthetic leaflet 570 may be modified in that a section of the prosthetic leaflet 570 may be pinched, folded, or otherwise gathered, to form a gathered section 575. For example, a length of material forming the prosthetic leaflet 570 may be gathered so that two portions of the same surface (e.g. either the surface that will face radially inwardly or radially outwardly upon formation of the prosthetic heart valve) contact or at least confront each other. The gathered section 575 may be fixed in the gathered configuration using a fixation mechanism 573. The fixation mechanism 573 may be any of those described herein in connection with other fixation mechanisms, including fixation mechanism 373 and 473, including for example a permanent or temporary fixation mechanism, and including for example an adhesive-type or binding-type mechanism, agent, or material.

As best shown in FIG. 12B, while gathered section 575 is maintained in the initial use condition, the free edge 572 maintains an effective length/distance L1 (which may also be referred to as an edge-to-edge length, a horizontal length, or a circumferential length), the effective length/distance L1 extending in a direction between the side edges 574 (and/or between the tabs 578) of the prosthetic leaflet 570. The effective length/distance L1 may be the maximum effective edge-to-edge length of the prosthetic leaflet 570 in this condition. When prosthetic leaflet 570 is coupled to a corresponding component of the prosthetic heart valve (e.g. an inner frame and/or inner skirt) and the prosthetic leaflet 570 has the initial use condition shown in FIGS. 12A-12B, the free edge 572 of the prosthetic leaflet 570 is relatively taut compared to the unmodified or final use condition, described in greater detail below in connection with FIGS. 12C-12D. As a result, while the prosthetic leaflet 570 is in the initial use condition, the free edge 572 of the prosthetic leaflet 570 is limited in how far radially inward the free edge 572 may extend when pressure on the outflow side of the prosthetic leaflet 570 is greater than the pressure on the inflow side of the prosthetic leaflet 570. This intentional limitation on the ability of the free edge 572 to fully extend radially inwardly will, in various embodiments, hinder sufficient coaptation with the free edges of other prosthetic leaflets to fully prevent backflow through the valve assembly when the prosthetic leaflets are in the closed condition. If the fixation mechanism 573 is a permanent (or substantially permanent) fixation mechanism, the regurgitation may be permanent (or substantially permanent), which, as described above, may be tolerated in some situations while avoiding potential harm from immediately resolving all regurgitation. If the fixation mechanism 573 is temporary, over time, the fixation mechanism 573 transitions (e.g. dissolves, degrades, releases, or absorbs) to allow for an increase in the effective length/distance of the free edge 572 (and thus a change in the extent to which the prosthetic leaflet 570 is able to extend radially inwardly during the coaptation phase of the prosthetic heart valve).

Referring now in addition to FIGS. 12C-12D, FIGS. 12C-12D illustrate front and top views (corresponding to the front and top views of FIGS. 12A-12B, respectively) of the prosthetic leaflet 570 in a final use condition (which may be the similar to or the same as a pre-assembled or pre-use condition) in which the fixation mechanism 573 has completely or substantially completely transitioned (e.g. degraded, released, absorbed, or dissolved). As best seen by comparing FIG. 12D to its counterpart FIG. 12B, the gathered section 575 has fully or substantially fully separated or unfurled as the fixation mechanism 573 fully or substantially fully transitioned (e.g. degraded or dissolved), so that the gathered section 575 has largely or completely disassociated from the leaflet. As a result of this complete (or substantially complete) reversal of the prior gathering of the gathered section 575, the free edge 572 has elongated to an effective length/distance L2, which is larger/longer than the prior effective length/distance L1. The effective length/distance L2 may be the maximum effective edge-to-edge length of the prosthetic leaflet 570 in this condition. This increase in effective length results in a corresponding decrease in tautness of the free edge 572. In other words, with the increased effective length/distance L2, compared to effective length/distance L1, the free edge 572 of the prosthetic leaflet 570 is able to move farther radially inwardly toward the other free edge(s) of the other prosthetic leaflet(s) forming the prosthetic heart valve. When the free edge 572 has the increased effective length/distance L2, the prosthetic leaflet 570 has the ability to fully (or more fully) coapt with the other prosthetic leaflet(s), and thus fully or substantially fully prevent regurgitation through the prosthetic heart valve, or to otherwise reduce the amount of regurgitation compared to the initial condition.

As noted above, in some examples, the fixation mechanism 573 may be permanent so that there is no significant change in the shape of the prosthetic leaflet 570 over time that is attributable by performance of a fixation mechanism, and no intentional change in regurgitation over time of the prosthetic heart valve that incorporates the prosthetic leaflet 570. In such examples, the amount of material used to form the gathered section 575 may be selected to decrease the effective length/distance of the free edge 572 (e.g. by gathering a relatively large amount of material of the prosthetic leaflet 570 before fixing the gathered section) to achieve a relatively large amount of regurgitation, or decrease the effective length/distance of the free edge 572 (e.g. by gathering a relatively small amount of material of the prosthetic leaflet 570 before fixing the gathered section) to achieve a relatively small amount of regurgitation.

Further, in examples in which the fixation mechanism 573 is configured to be temporary, the transition (e.g. degradation, dissolution, release, absorption etc.) of the fixation mechanism 573 may be gradual, stepwise or substantially binary as also discussed above with respect to other examples. In other words, although two total use conditions are shown in FIGS. 12A-12B, the fixation mechanism 573 may degrade over time at a constant rate (or a non-constant rate) so that the change in the effective length/distance of the free edge 572 is gradual, allowing for progressively better coaptation and progressively smaller regurgitation until the regurgitation is completely (or substantially completely) resolved to a desired degree. In other examples, the fixation mechanism 573 may have more of a binary release. For example, most, substantially all, or all of the material forming the fixation mechanism 573 may be configured to transition (e.g. degrade, dissolve, absorb or release) at about the same time point such that, as soon as the transition occurs, the gathered section 575 may unfurl or release at once to achieve a relatively abrupt transition (e.g. from an initial use condition which may have a configuration similar to FIGS. 12A-12B) to the final condition similar to that shown in FIGS. 12C-12D.

In examples in which the fixation mechanism 573 is temporary, as with fixation mechanisms 373 and 473 described above, the particular fixation mechanism 573 may be selected in order to allow for regurgitation for a desired amount of time before the fixation mechanism 573 degrades or dissolves, including for example 1 week, 1 month, 6 months, 9 months, 1 year, etc. as also discussed above. It should be understood that the time periods noted above are merely exemplary.

Further, the prosthetic heart valve that incorporates prosthetic leaflet 570 may include only one prosthetic leaflet 570 with a gathered section 575, or more than one such prosthetic leaflet 570. Although FIG. 12A shows the gathered section 575 being initially positioned along a full axial height of the prosthetic leaflet 570 (e.g. completely extending from the free edge 572 to the attached edge 576) and being positioned generally closer to one side edge 574 of the prosthetic leaflet 570, it should be understood that this positioning is merely exemplary. For example it may be desirable for the attached edge 576 to be excluded from the gathered section 575, as that edge may already serve as an attachment point to another component of the prosthetic heart valve (e.g. a frame and/or inner cuff). Similarly, in some examples, whether the gathered section 575 extends to the attached edge 576 or not, the gathered section 575 may stop short of the free edge 572. In such examples, the effective length/distance of the free edge 572 may or may not be changed, but the prosthetic leaflet 570 may still have an increased level of tautness while the gathered section 575 exists. In other examples, the gathered section 575 may have a greater or smaller height than shown, and may be positioned at different locations (e.g. closer to either side edge 574 and/or closer to center between the side edges 574) than shown in the figures. Still further, it should be understood that, although prosthetic leaflet 570 is shown as having a single gathered section 575, a single prosthetic leaflet 570 may instead include two or more gathered sections 575. Further, it should be understood that a prosthetic leaflet may include both one or more gathered section 575 along with a modified free edge portion similar to that shown in FIGS. 10A-B. Further, a prosthetic heart valve that includes two or more leaflets may include one or more prosthetic leaflets having any of the configurations (including combinations) described above along with one or more additional prosthetic leaflets having the same configuration or any of the other configurations described above to fine-tune the amount of regurgitation, as well as the time profile by which the regurgitation reduces. As described in greater detail below, this optionality includes using different fixation mechanisms for each gathered portion and/or modified free edge with different time-release profiles, such that the individual gathered portions or modified free edges unfurl or release at different times.

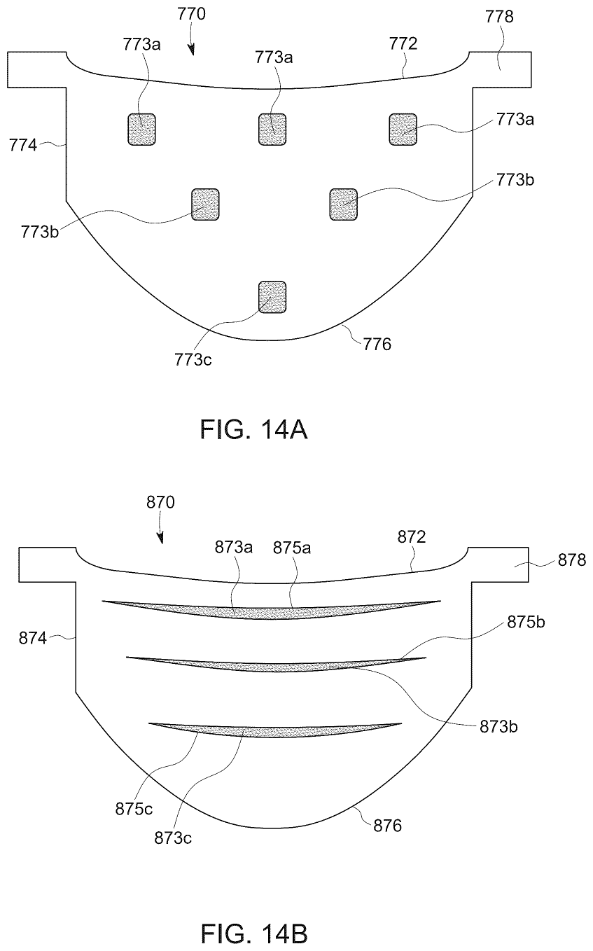

Although the examples described in connection with FIGS. 10A-12D generally include a prosthetic leaflet having an initial use condition in which the prosthetic leaflet is coupled to itself to cause a geometry change in the prosthetic leaflet to induce, at least temporarily, regurgitation, it should be understood that similar results may be achieved instead by coupling a portion of a prosthetic leaflet to another component of the prosthetic heart valve. For example, referring now in addition to FIG. 13A, FIG. 13A shows a top view of a portion of a prosthetic heart valve 600. It should be understood that prosthetic heart valve 600 is shown as a largely generic prosthetic heart valve with three prosthetic leaflets 670a, 670b, 670c. It should be understood that the disclosure below with respect to prosthetic leaflet 670a may be applied to prosthetic heart valves of various different types, including for example prosthetic heart valves 100 or 200 described above. Thus, other than the disclosure regarding the use of fixation mechanisms 673a to at least temporarily induce regurgitation in a prosthetic heart valve, the remaining components of prosthetic heart valve 600 should be understood be exemplary in nature.

In the example prosthetic heart valve 600 schematically depicted in FIG. 13A, a generally cylindrical frame 610 is provided with three commissure attachment features 650 at substantially equal intervals, and three corresponding prosthetic leaflets 670a, 670b, 670c are provided. Each prosthetic leaflet 670a-670c may have a pre-use (i.e. pre-implantation) condition that is generally similar in shape to the pre-use condition of any of the other prosthetic leaflets described herein. For example, each prosthetic leaflet 670a, 670b, 670c may include a corresponding free edge 672a, 672b, 672c adapted to come together during a coaptation phase of the prosthetic heart valve 600. It should be understood that FIGS. 13A-13B each show a coaptation phase of the prosthetic heart valve 600. Each prosthetic leaflet 670a-670c may include a pair of side edges and/or tabs (e.g. similar to those described above for other prosthetic leaflets herein). A first side edge and/or tab of a first one of the prosthetic leaflets may be joined to the frame 610 at a first commissure attachment feature 650 to which a first side edge and/or tab of a second one of the prosthetic leaflets is joined. In other words, each commissure attachment feature 650 may provide for attachment of one end of a one prosthetic leaflet and another end of another prosthetic leaflet.

Referring to FIG. 13A, a portion of the free edge 672a of prosthetic leaflet 670a may be coupled to another component of the prosthetic heart valve 600, such as frame 610. In other words, a portion of the free edge 672a that would typically be available for coaptation with another one of the prosthetic leaflets (e.g. prosthetic leaflet 670c in FIG. 13A) may instead be restricted from movement via coupling to another component of the prosthetic heart valve 600. In the illustrated example, the portion of the free edge 672a that is coupled to the frame 610 is a length of the free edge 672a that is adjacent to where the corresponding side or tab of the prosthetic leaflet 670a is coupled to the commissure attachment feature 650. The coupling may be achieved via a fixation mechanism 673a, which may be similar or identical to any of the other fixation mechanisms (e.g. fixation mechanisms 373, 473, or 573) described herein. Similar to other embodiments described herein, the fixation mechanism 673a may be permanent or temporary to induce permanent or temporary regurgitation through the prosthetic heart valve 600. As shown in FIG. 13A, as pressure on the outflow side of the prosthetic heart valve 600 increases compared to pressure on the inflow side of the prosthetic heart valve 600, the free edges 672a, 672b, 672c are pushed toward each other in a coaptation phase of the prosthetic heart valve 600. However, because a portion of the free edge 672a of prosthetic leaflet 670 is at least temporarily fixed to frame 610, that portion is unable to move radially inwardly to coapt with another prosthetic leaflet (e.g. prosthetic leaflet 670c) of the prosthetic heart valve 600. The result is that, in the coaptation phase of the prosthetic heart valve 600, a gap between the prosthetic leaflets 670a-670c remains, so that regurgitant flow RF of blood is induced through the prosthetic heart valve 600.

In examples in which fixation mechanism 673a is temporary (e.g. via gradual dissolution, stepwise or binary release), transition (e.g. absorption, degradation, release or dissolution, etc.) of the fixation mechanism 673a frees the previously-restricted portion of the free edge 672a for coaptation. For example, FIG. 13B shows the prosthetic heart valve 600 in the coaptation phase after the fixation mechanism 673a has completely or substantially completely transitioned (e.g. dissolved or released). In this condition, which may be considered a final use condition, the free edge 672a of prosthetic leaflet 670a is capable of fully coapting with the free edges 672b, 672c of prosthetic leaflets 670b, 670c so that no gap is present between the free edges, fully or substantially fully eliminating the prior-existing zone of regurgitant flow RF. However, it should be understood that, if desired, the final use condition may retain some amount of intended regurgitant flow RF, which may be smaller than the amount of intended regurgitant flow RF in the initial condition.

Regarding the actual composition of the fixation mechanism 673a, including options for materials, the temporary or permanent nature of the fixation, and the different time options of dissolution or release for temporary versions of the fixation mechanism 673a, the options described in connection with the other fixation mechanisms herein apply fully to fixation mechanism 673a. Further, although the free edge 672a is shown as being fixed to the frame 610, it may instead or additionally be fixed to other components of the prosthetic heart valve 600 that would restrict the ability for the prosthetic leaflet 670a to fully coapt with other prosthetic leaflets, such as to an inner skirt and/or to a commissure ring (such as commissure support ring 180 or 280). It should be understood that, although the terminology used above describes a portion of the free edge 672a of the prosthetic leaflet 670a being attached to another component of the prosthetic heart valve 600, that attached portion would, at least as long as the fixation mechanism 673a is present, not actually be “free” to move. Thus, it should be understood that when it is described that a portion of the free edge 672a is attached to another component of the prosthetic heart valve 600, that portion should be understood as a free edge in the sense that, but for the fixation mechanism 673a, that portion would be free to coapt with the other prosthetic leaflets.

Although the example FIG. 13A includes fixation of a portion of the free edge 672a of the prosthetic leaflet 670 to another component of the prosthetic heart valve 600, it should be understood that a different portion of the prosthetic leaflet 670 may instead or additionally be fixed, at least temporarily, to another component of the prosthetic heart valve 600 to intentionally induce regurgitation through the prosthetic heart valve 600. For example, any portion of the prosthetic leaflet 670a (including portions adjacent the attached edge, any portion of the free edge, or any portion of the leaflet therebetween) may be at least temporarily fixed to another component of the prosthetic heart valve 600 in order to restrict the ability of the prosthetic leaflet 670a to fully coapt with the other prosthetic leaflets 670b, 670c for inducing a zone of regurgitant flow RF during the coaptation phase of the prosthetic heart valve 600.

Although prosthetic heart valve 600 is shown with a single leaflet 670a including a fixation mechanism 673a to restrict full leaflet coaptation, it should be understood that more than one of the prosthetic leaflets may include such a fixation mechanism in a similar manner. Further, although the fixation mechanism 673a is shown in FIG. 13A as being at a single area on the free edge 672a adjacent to where the prosthetic leaflet 670a attached to a commissure attachment feature 650, multiple areas of the prosthetic leaflet 670a may be coupled to another component of the prosthetic heart valve 600 to restrict completeness of coaptation. For example, a second fixation mechanism may be provided near where the free edge 672a attaches to the other commissure attachment feature 650 to potentially increase the size of the zone of regurgitant flow RF. Further, any of the options for inducing regurgitation described in connection with FIGS. 13A-B may be combined with any of the options for inducing regurgitation described in connection with FIGS. 10A-12D.

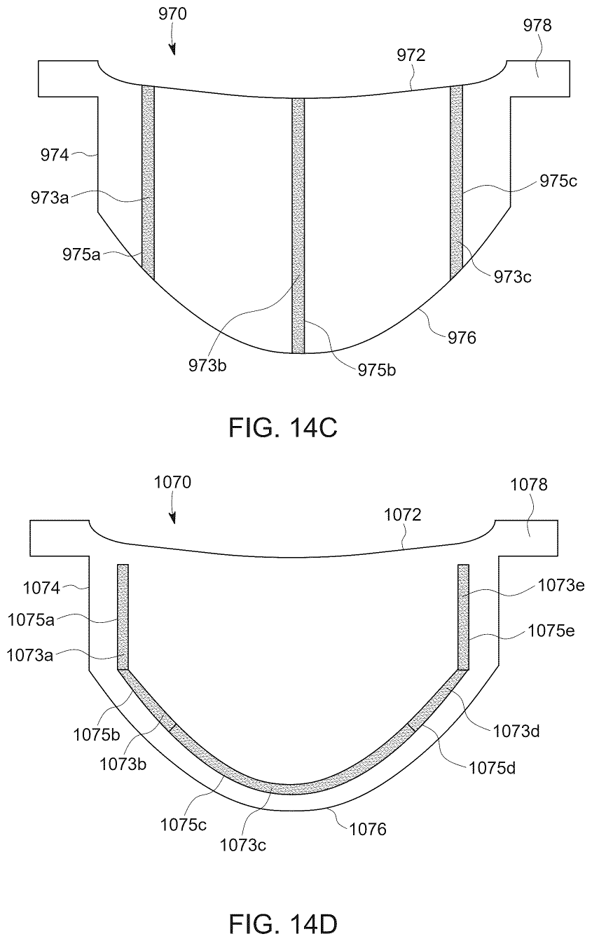

Although the various embodiments of prosthetic leaflets described above are generally shown in the particular illustrated embodiments with a single leaflet modification that induces regurgitation, as also described in connection with those embodiments, a single prosthetic leaflet may include more than one such modification. FIGS. 14A-14D illustrate various examples of prosthetic leaflets that each have two or more such modifications.

Referring now in addition to FIG. 14A, FIG. 14A schematically illustrates an example of a prosthetic leaflet 770, as if flattened and laid out on a table. Prosthetic leaflet 770 may be generally similar or identical to the other prosthetic leaflets described herein, with certain exceptions relating to the particular regurgitation-inducing modification(s). Thus, other than stating that the prosthetic leaflet 770 may include a free edge 772, an attached edge 776, and side edges 774 (and/or leaflet tabs 778), other aspects of the prosthetic leaflet 770 that are similar or identical to the other prosthetic leaflets described herein are not described in further detail again here. Prosthetic leaflet 770 may include a plurality of modifications which may may function to at least temporarily restrict coaptation or to at least temporarily induce regurgitation. In the specific illustrated embodiment, prosthetic leaflet 770 includes a plurality of fixation mechanisms arranged in rows, including three fixation mechanisms 773a positioned near the free edge 772, two fixation mechanisms 773b positioned in a row between the fixation mechanisms 773a and the attached edge 776, and one fixation mechanism 773c positioned between the fixation mechanisms 773b and the attached edge 776. It should be understood that the illustrated placement of the rows, the illustrated number of rows, the illustrated number of fixation mechanisms per row, and the illustrated relative placement of fixation mechanisms in each row is exemplary, and deviations may achieve similar results. In this example, the fixation mechanisms may each be applied to portions of the prosthetic leaflet 770 that are gathered together (e.g. with a horizonal fold/pinch generally similar to that shown and described in connection with FIG. 11A-1, with a vertical fold/pinch generally similar to that shown and described in connection with FIG. 12A, or any other way in which an area of the prosthetic leaflet 770 may be gathered) and then fixed in the gathered state. As with the fixation mechanisms of other embodiments, these fixation mechanisms 773a-773c may be applied on a luminal surface of the prosthetic leaflet, an abluminal surface of the prosthetic leaflet, on both surfaces, or combinations thereof, including some on one surface and some on the other surface. Alternatively, instead of being a gathered portion of the prosthetic leaflet 770, fixation mechanisms 773a-773c may be applied to portions of the prosthetic leaflet 770 that are fixed to another component of the prosthetic heart valve, such as a frame and/or inner skirt, generally similar to as described in connection with FIG. 13A. In some examples, one or more of the fixation mechanisms 773a-773c may be applied to portions of the leaflet that are gathered, and one or more of the fixation mechanisms 773a-773c may be applied to the prosthetic leaflet 770 to fix portions of the prosthetic leaflet 770 to another component of the prosthetic heart valve such as a frame and/or inner cuff. In some examples, the fixation mechanisms 773a-773c may be applied to relatively small areas of the prosthetic leaflet, for example compared to the fixation mechanisms 473, 573 applied to gathered sections 475, 575.

In some examples, all of the fixation mechanisms 773a-773c may be formed using the same type of fixation mechanism (e.g. the same adhesive material). If such a fixation mechanism 773a-773c is temporary, each of the portions of the leaflet to which the fixation mechanism 773a-773c is applied may unfurl or release as the fixation mechanism transitions (e.g. dissolves, degrades, absorbs, or otherwise releases), such that the fixation mechanisms 773a-773c all disassociate from the leaflet 770 or stop functioning to restrict coaptation at a generally similar time. However, in other examples, the fixation mechanisms 773a-773c may be formed using different types of fixation mechanisms (e.g. different adhesives) configured to transition (e.g. degrade or dissolve) at different rates. For example, the outflow row of fixation mechanisms 773a may be formed using a material configured to dissolve or degrade after a first time period of being implanted (e.g. about 6 months), the middle row of fixation mechanisms 773b may be formed using a material configured to dissolve or degrade after a second time period shorter than the first time period (e.g. about 3 months), and the inflow row (in the illustrated example being a row having a single member) of fixation mechanisms 773c may be formed using a material configured to dissolve or degrade after a third time period shorter than the second time period (e.g. about 1 month). It should be understood that these time periods are merely exemplary, and other time periods with the same relative transition (e.g. degradation or dissolution) rates may be used, as desired. However, other relative transition (e.g. dissolution or degradation) rates may also be used, for example with the outflow row of fixation mechanisms 773a having a relatively short time before dissolution or degradation, the inflow row of fixation mechanisms 773c having a relatively long time before dissolution or degradation, and the middle row of fixation mechanisms 773b having an intermediate time before dissolution or degradation. In the examples in which the various fixation mechanisms 773a-773c are configured to transition (e.g. dissolve or degrade) after different lengths of time after implantation, the prosthetic leaflet 770 may become progressively less restricted from achieving good coaptation with the other prosthetic leaflets over time as more and more of the prosthetic leaflet 770 becomes free to move as intended into contact with the other prosthetic leaflets.

Referring now in addition to FIG. 14B, FIG. 14B illustrates another example of a prosthetic leaflet 870, as if flattened and laid out on a table. Prosthetic leaflet 870 may be generally similar or identical to the other prosthetic leaflets described herein, with certain exceptions relating to the particular regurgitation-inducing modification(s). Thus, other than stating that the prosthetic leaflet 870 may include a free edge 872, an attached edge 876, and side edges 874 (and/or leaflet tabs 878), other aspects of the prosthetic leaflet 870 that are similar or identical to the other prosthetic leaflets described herein are not described in further detail again here. Prosthetic leaflet 870 is generally similar to the example of prosthetic leaflet 470 shown in FIG. 11A-1, with the main exception being that prosthetic leaflet 870 is shown with three fixation mechanisms 873a-873c, resulting in corresponding gathered sections 875a-875c, instead of a single gathered section 475. The particular embodiment shows three gathered sections 875a, 875b, 875c spaced at about equal intervals in the inflow-to-outflow direction and being generally centered between the side edges 874, although two or more than three gathered sections may be provided. In the illustrated examples, the fixation mechanisms 873a-873c are all applied to the same side of the prosthetic leaflet 870 that is visible in FIG. 14B, although the fixation mechanisms 873a-873c may all be provided on the other side of the prosthetic leaflet 870, or with some on one side of the prosthetic leaflet 870 and others on the other side of the prosthetic leaflet 870. In some examples, the fixation mechanisms 873a-873c that maintain the gathered sections 875a-875c in the gathered condition may all be the same such that the gathered sections 875a-875c all release or unfurl at about the same time. However, in other examples, the fixation mechanisms 873a-873c among the various gathered sections 875a-875c may be formed using different fixation mechanisms configured to degrade at different rates. For example, the top or outflow gathered section 875a may be formed using a fixation mechanism 873a configured to transition (e.g. dissolve or degrade) after a first time period of being implanted (e.g. about 6 months), the middle gathered section 875b may be formed using a fixation mechanism 873b configured to transition (e.g. dissolve or degrade) after a second time period shorter than the first time period (e.g. about 3 months), and the bottom or inflow gathered section 875c may be formed using a fixation mechanism 873c configured to transition (e.g. dissolve or degrade) after a third time period shorter than the second time period (e.g. about 1 month). It should be understood that these time periods are merely exemplary, and other time periods with the same relative transition (e.g. degradation or dissolution rates) may be used as desired. However, other relative transition (e.g. dissolution or degradation) rates may also be used, for example with the top or outflow gathered section 875a having a relatively short time before dissolution or degradation of the fixation mechanism 873a, the bottom or inflow gathered section 875c having a relatively long time before dissolution or degradation of the fixation mechanism 873c, and the middle gathered section 875b having an intermediate time before dissolution or degradation of the fixation mechanism 873b. In the examples in which the fixation mechanisms 873a-873c of the various gathered sections 875a-875c are configured to dissolve or degrade after different lengths of time after implantation, the prosthetic leaflet 870 may become progressively less restricted from achieving good coaptation with the other prosthetic leaflets over time as more and more of the prosthetic leaflet 870 becomes free to move as intended into contact with the other prosthetic leaflets or otherwise reduce regurgitation as compared to the initial state upon implantation. Further, although gathered sections 875a-875c are described as being similar to gathered section 475 in which the prosthetic leaflet is coupled to itself, one, some or all of the gathered sections 875a-875c may instead be formed more similar to the example of FIG. 13A, for example by fixing the relevant portion of the prosthetic leaflet to another component of the prosthetic heart valve using the fixation mechanisms 873a-873c, such as the frame and/or inner cuff.

Referring now in addition to FIG. 14C, FIG. 14C illustrates a further example of a prosthetic leaflet 970, as if flattened and laid out on a table. Prosthetic leaflet 970 may be generally similar or identical to the other prosthetic leaflets described herein, with certain exceptions relating to the particular regurgitation-inducing modification(s). Thus, other than stating that the prosthetic leaflet 970 may include a free edge 972, an attached edge 976, and side edges 974 (and/or leaflet tabs 978), other aspects of the prosthetic leaflet 970 that are similar or identical to the other prosthetic leaflets described herein are not described in further detail again here. Prosthetic leaflet 970 is generally similar to the example of prosthetic leaflet 570 shown in FIG. 12A, with the main exception being that prosthetic leaflet 970 is shown with three fixation mechanisms 973a-973c, each resulting in a corresponding gathered section 975a-975c, instead of a single gathered section 575. The particular embodiment shows three gathered sections 975a, 975b, 975c extending in an axial or inflow-to-outflow direction and spaced at about equal intervals in the circumferential direction between the side edges 974, although two or more than three gathered sections may be provided. In the illustrated examples, the fixation mechanisms 973a, 973b, 973c are all applied to the same side of the prosthetic leaflet 970 that is visible in FIG. 14C, although the fixation mechanisms 973a-973c may all be provided on the other side of the prosthetic leaflet 970, or with some on one side of the prosthetic leaflet 970 and others on the other side of the prosthetic leaflet 970. In some examples, the fixation mechanisms 973a-973c that maintain the gathered sections 975a-975c in the gathered condition may all be the same such that the gathered sections 975a-975c all release or unfurl at about the same time. However, in other examples, the fixation mechanisms 973a-973c among the various gathered sections 975a-975c may be formed using different fixation mechanisms configured to degrade at different rates. For example, the left-most gathered section 975a may be formed using a fixation mechanism 973a configured to transition (e.g. dissolve or degrade) after a first time period of being implanted (e.g. about 6 months), the middle gathered section 975b may be formed using a fixation mechanism 973b configured to transition (e.g. dissolve or degrade) after a second time period shorter than the first time period (e.g. about 3 months), and the right-most gathered section 975c may be formed using a fixation mechanism 973c configured to transition (e.g. dissolve or degrade) after a third time period shorter than the second time period (e.g. about 1 month). It should be understood that these time periods are merely exemplary, and other time periods with the same relative transition (e.g. degradation or dissolution) rates may be used. However, other relative transition (e.g. dissolution or degradation) rates may also be used, for example with the fixation mechanism 973a on the left-most gathered section 975a having a relatively short time before dissolution or degradation, the fixation mechanism 973c on the right-most gathered section 975c having a relatively long time before dissolution or degradation, and the fixation mechanism 973b on the middle gathered section 975b having an intermediate time before dissolution or degradation. Still other examples are possible, including a relatively long time to dissolution for the fixation mechanism 973b on the middle gathered section 975b, and relatively short times to dissolution for the fixation mechanisms 973a, 973c on the left-most and right-most gathered sections 975a, 975c, or in other examples a relatively short time to dissolution for the fixation mechanism 973b on the middle gathered section 975b, and relatively long times to dissolution for the fixation mechanisms 973a, 973c on the left-most and right-most gathered sections 975a, 975c. In the examples in which the fixation mechanisms 973a-973c on the various gathered sections 975a-975c are configured to dissolve or degrade after different lengths of time after implantation, the prosthetic leaflet 970 may become progressively less restricted from achieving good coaptation with the other prosthetic leaflets over time as more and more of the prosthetic leaflet 970 becomes free to move as intended into contact with the other prosthetic leaflets. Further, although gathered sections 975a-975c are described as being similar to gathered section 575 in which the prosthetic leaflet is coupled to itself by the fixation mechanism(s), one, some or all of the gathered sections 975a-975c may instead be formed more similar to the example of FIG. 13A, for example by fixing the relevant portion of the prosthetic leaflet to another component of the prosthetic heart valve via fixation mechanisms 973a-973c, such as the frame and/or inner cuff.

Referring now in addition to FIG. 14D, FIG. 14D illustrates another example of a prosthetic leaflet 1070, as if flattened and laid out on a table. Prosthetic leaflet 1070 may be generally similar or identical to the other prosthetic leaflets described herein, with certain exceptions relating to the particular regurgitation-inducing modification(s). Thus, other than stating that the prosthetic leaflet 1070 may include a free edge 1072, an attached edge 1076, and side edges 1074 (and/or leaflet tabs 1078), other aspects of the prosthetic leaflet 1070 that are similar or identical to the other prosthetic leaflets described herein are not described in further detail again here. Prosthetic heart valve 1070 may include one or more fixation mechanisms 1073a-1073e. Although FIG. 14D illustrates five individual fixation mechanisms 1073a, 1073b, 1073c, 1073d, 1073e, it should be understood that these fixation mechanisms 1073a-1073e may form a single continuous fixation mechanism. As illustrated, the fixation mechanisms 1073a-1073e are arranged to form a generally “U” shaped pattern that generally follows the contour of the attached edge 1076 but which may be a spaced distance from the attached edge 1076. In some examples, the fixation mechanisms 1073a-1073e may be applied to the prosthetic leaflet 1070 to maintain gathered sections 1075a-1075e of the prosthetic leaflet 1070 in a gathered condition, for example with vertical gathered sections 1075a and 1075e (which may be similar to gathered section 575), and diagonal or curved gathered sections 1075b, 1075c, 1075d extending between the gathered sections 1075a, 1075e. In other examples, the fixation mechanisms 1073a-1073e may be applied to the prosthetic leaflet 1070 to attach the relevant sections of the prosthetic leaflet 1070 to another component of the prosthetic heart valve to restrict coaptation, such as the frame and/or inner skirt, generally similar the example described in connection with FIG. 13A.

Regardless of the specific number, position, and way in which the fixation mechanisms 1073a-1073e are applied, each fixation mechanism 1073a-1073e may be similar to any of the fixation mechanisms described above. In some examples, all or a plurality of the fixation mechanisms 1073a-1073e may be the same. If such a fixation mechanism is temporary, each of the gathered sections 1075a-1075e (or otherwise the relevant portions of the leaflet that are attached to the stent and/or cuff) may unfurl or release as the fixation mechanisms 1073a-1075e transitions (e.g. dissolves, degrades, absorbs, or otherwise releases), such that the fixation mechanisms 1073a-1073e all disassociate from the leaflet 1070 or otherwise stop functioning to restrict coaptation at a generally similar time. However, in other examples, the fixation mechanisms 1073a-1073e may be formed using different fixation mechanisms configured to transition (e.g. degrade or dissolve) at different rates. For example, the vertical or outer-most fixation mechanisms 1073a, 1073e may be formed using a material configured to dissolve or degrade after a first time period of being implanted (e.g. about 6 months), the intermediate fixation mechanisms 1073b, 1073d may be formed using a material configured to dissolve or degrade after a second time period shorter than the first time period (e.g. about 3 months), and the middle or center fixation mechanism 1073c may be formed using a material configured to dissolve or degrade after a third time period shorter than the second time period (e.g. about 1 month). It should be understood that these time periods are merely exemplary, and other time periods with the same relative transition (e.g. degradation or dissolution) rates may be used, as desired. However, other relative transition (e.g. dissolution or degradation) rates may also be used, for example with the vertical or outer-most fixation mechanisms 1073a, 1073e having a relatively short time before dissolution or degradation, the middle or center fixation mechanism 1073c having a relatively long time before dissolution or degradation, and the intermediate fixation mechanisms 1073b, 1073d having an intermediate time before dissolution or degradation. In the examples in which the various fixation mechanisms 1073a-1073e are configured to dissolve or degrade after different lengths of time after implantation, the prosthetic leaflet 1070 may become progressively less restricted from achieving good coaptation with the other prosthetic leaflets over time as more and more of the prosthetic leaflet 1070 becomes free to move as intended into contact with the other prosthetic leaflets.

As described elsewhere herein, various different individual mechanisms of at least temporarily restricting leaflet coaptation and/or at least temporarily inducing regurgitation in a prosthetic heart valve are described above. Although described as individual examples, it should be understood that any one example of a mechanism for restricting coaptation may be combined with any other mechanism for restricting coaptation, which may help to fine-tune the profile with which regurgitation reduces over time. This includes combining temporary fixation mechanisms with permanent fixation mechanisms, as well as combining the use of gathered sections of prosthetic leaflets that are coupled together with sections of prosthetic leaflets that are coupled to other components of the prosthetic heart valve to affect leaflet motion. Further, although the examples described as having gathered sections are generally described as achieving coaptation restriction by coupling the prosthetic leaflet to itself upon folding or gathering, these examples may instead achieve coaptation restriction by coupling the relevant portions of the prosthetic leaflet to another component of the prosthetic heart valve, such as a frame or inner skirt. Further, although the terminology of “gathered section” is typically used herein in connection with coupling one portion of a prosthetic leaflet to another portion of the prosthetic leaflet, and different terminology may be used when describing sections of the prosthetic leaflet being coupled to a frame or inner skirt (or any other component of the prosthetic heart valve that would serve to limit free motion of the prosthetic leaflet), it should be understood that these features may all generically be described as coaptation restrictors and/or regurgitation inducers. Still further, although some embodiments, such as prosthetic leaflet 470, describes generally horizontal or circumferential pinching or folding or gathering to reduce the effective leaflet axial height/distance, and other embodiments, such as prosthetic leaflet 570, describes generally vertical or axial pinching or folding or gathering to increase the tautness of the prosthetic leaflet and/or to reduce the effective length/distance of the free edge of the leaflet, it should be understood that these configurations may generically be described as modifying, at least temporarily, the geometry, distance (e.g. maximum distance in a particular condition) and/or dimensions of the prosthetic leaflet to limit the ability of the prosthetic leaflet to move through the full intended range of motion radially inwardly toward other prosthetic leaflets of the prosthetic heart valve. In addition to at least temporarily reducing the maximum effective length and/or maximum effective height of the leaflet, the fixation mechanisms may at least temporarily reduce the effective surface area of the leaflet (e.g. the area of the leaflet available to move toward the other leaflets for coapting). If the fixation mechanism is temporary, upon transitioning (e.g. dissolving or degrading) after a period of time in situ, the maximum effective length and/or effective height and/or effective surface area of the leaflet may increase, which may lead to better coaptation and less regurgitation.

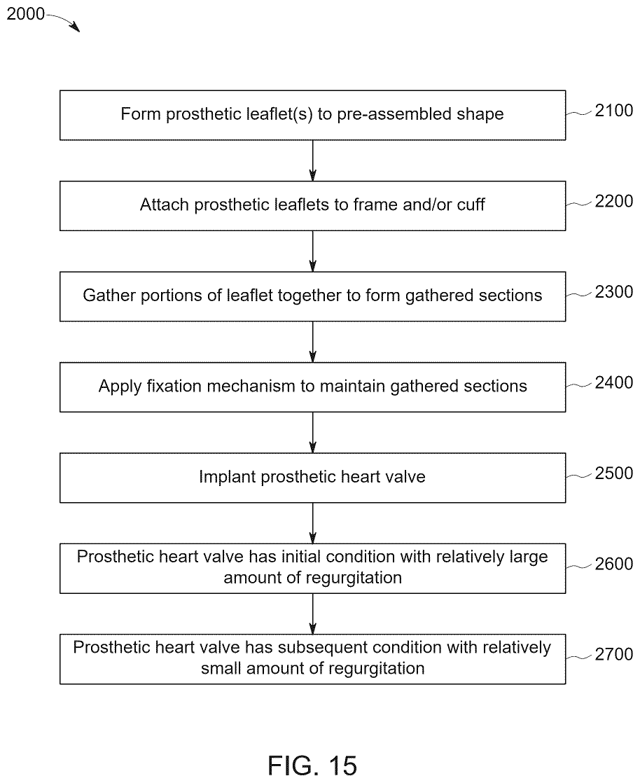

FIG. 15 shows example steps 2100-2700 of an example method 2000 of manufacturing and/or using a prosthetic heart valve of any of the types disclosed herein. In example step 2100, one or more prosthetic leaflets of the disclosure may be formed into a pre-use or pre-assembled shape. This may include, for example, forming a fabric and cutting the fabric to the desired shape, or obtaining tissue (e.g. porcine or bovine pericardial tissue) and cutting the tissue to the desired shape. In some examples, the pre-assembled shape of the prosthetic leaflet(s) may be similar to the shape of prosthetic leaflet 270 shown in FIG. 9. In some examples, including those in which the prosthetic leaflet is a tissue leaflet, step 2100 may include treating the tissue, for example by fixing the tissue with glutaraldehyde. After the prosthetic leaflet(s) is formed, in step 2200 the prosthetic leaflet(s) may be attached to a frame and/or to a skirt. For example, tabs and/or side edges of the prosthetic leaflet(s) may be coupled to a frame such as frame 110, 210, or 610 via commissure attachment features 150, 250, or 650. In some examples, the attached edge of the prosthetic leaflet(s) may be coupled, for example via suturing, to an inner skirt positioned between the prosthetic leaflet(s) and the frame. In some examples, a commissure support ring, such as commissure support ring 180 or 280 may be coupled to the outer surface of commissure attachment features to provide additional support. In step 2300, portions of one or more of the prosthetic leaflets may be gathered, for example by folding, pinching, or the like, and in step 2400, one or more fixation mechanisms of the disclosure may be applied to the prosthetic leaflet(s) at the gathered portions to at least temporarily maintain the gathered portions in the gathered condition. The configurations for gathered portions include, but are not limited to, examples described in connection with FIGS. 10A-12D and FIGS. 14A-14D. The fixation mechanism(s) applied in step 2400 may include, but are not limited to, any fixation mechanisms described herein, including temporary or permanent fixation mechanisms, including for example adhesive-type or binding-type fixation mechanisms. If the fixation mechanism(s) is temporary, the particular fixation mechanism(s) may be selected to transition (e.g. degrade, dissolve, absorb or otherwise release) after a desired time point after implantation. In some examples, although the gathering step 2300 and fixation step 2400 are described as being performed after attaching the prosthetic leaflet(s) to the frame and/or cuff, in other examples such steps may be performed before step 2200 or simultaneously with step 2200. After the prosthetic heart valve is fully assembled, it may be implanted into a patient in step 2500. Upon implantation of the assembled prosthetic heart valve, regurgitation may be (but need not be) reduced compared to the amount of regurgitation existing in the native valve just prior to implantation, but at least some amount of regurgitation across the prosthetic heart valve may be intentionally maintained. This initial condition with a first level of regurgitation is represented by step 2600. As described in greater detail above, such intentional regurgitation may be permanent, substantially permanent, or may be temporary, with either a substantially binary change, step-wise change, or graduated change after implantation from the intentional amount of regurgitation to the complete or substantially complete elimination of regurgitation (or to some other intentionally designed level of regurgitation). Thus, in some examples, the prosthetic heart valve transitions to a subsequent condition, represented by step 2700, in which the regurgitation across the prosthetic heart valve is relatively small compared to that found in step 2600. In some examples, the subsequent condition may be a final condition with no regurgitation, substantially no regurgitation, or some other intentionally designed level of regurgitation. In other examples, the subsequent condition may be an intermediate condition with lower regurgitation than the initial condition, with the prosthetic heart valve later transitioning to a final condition with relatively low (including no) regurgitation.

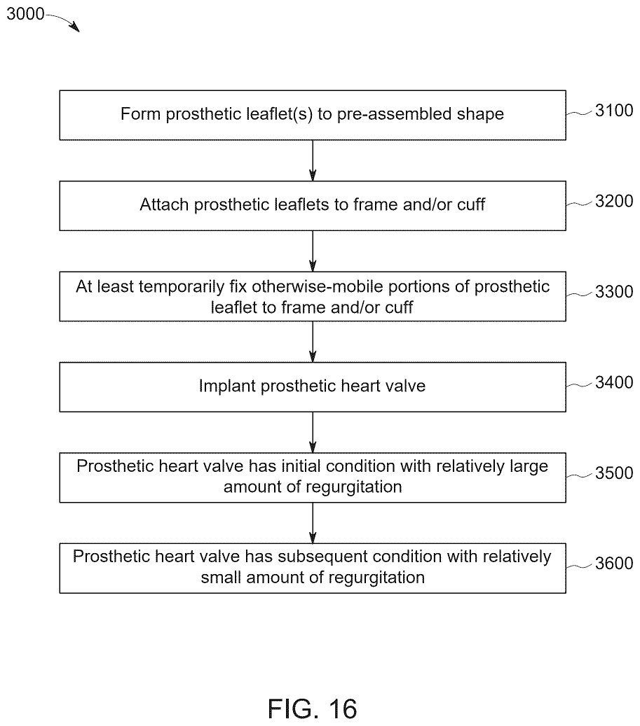

FIG. 16 shows example steps 3100-3600 of another example method 3000 of manufacturing and/or using a prosthetic heart valve of the disclosure. In example step 3100, one or more prosthetic leaflets may be formed into a pre-used or pre-assembled shape, similar or identical to as described above for step 2100. After the prosthetic leaflet(s) is formed, in step 3200 the prosthetic leaflet(s) may be attached to a frame and/or to a skirt, similar or identical to as described above for step 2200. In step 3300, portions of one or more of the prosthetic leaflet(s) that would otherwise be mobile after implantation may be attached (e.g. using a fixation mechanism including, but not limited to, any of those described above) to another component of the prosthetic heart valve to at least temporarily restrict or reduce the mobility of the prosthetic leaflet(s). For example, portions of one or more of the leaflet(s) may be attached to the inner cuff and/or frame similar to that shown and described in connection with FIG. 13A, and/or portions described as being gathered in connection with FIGS. 11A-1 through 12D and FIGS. 14A through 14D may instead be attached to the frame and/or inner skirt instead of being gathered. The fixation mechanism(s) applied in step 3300 may include, but are not limited to, any fixation mechanisms described herein, including temporary or permanent or substantially permanent fixation mechanisms, including for example adhesive-type or binding-type fixation mechanisms. If the fixation mechanism(s) is temporary, the particular fixation mechanism(s) may be selected to transition (e.g. degrade, dissolve, absorb, otherwise release, etc.) after a desired time point after implantation. After the prosthetic heart valve is fully assembled, it may be implanted into a patient in step 3400. Upon implantation of the assembled prosthetic heart valve, regurgitation may be (but need not be) reduced compared to the amount or regurgitation existing in the native valve just prior to implantation, but at least some amount of regurgitation across the prosthetic heart valve may be intentionally maintained. This initial condition with a first level of regurgitation is represented by step 3500. As described in greater detail above, such intentional regurgitation may be permanent, or may be temporary, with either a substantially binary change, step-wise change, or graduated change after implantation from the intentional amount of regurgitation to the complete or substantially complete elimination of regurgitation (or to some other intentionally designed level of regurgitation). Thus, in some examples, the prosthetic heart valve transitions to a subsequent condition, represented by step 3600, in which the regurgitation across the prosthetic heart valve is relatively small compared to that found in step 3500. In some examples, the subsequent condition may be a final condition with no regurgitation, substantially no regurgitation, or some other intentionally designed level of regurgitation. In other examples, the subsequent condition may be an intermediate condition with lower regurgitation than the initial condition, with the prosthetic heart valve later transitioning to a final condition with relatively low (including no) regurgitation.

Finally, although methods 2000 and 3000 are presented as different methods, it should be understood that the methods may be combined (for example steps 2300 and 2400 may be done along with step 3300, in the context of a single method using steps 2100 (or identical step 3100), step 2200 (or identical step 3200), and step 2500 (or identical step 3400)). In other words, a prosthetic heart valve may be assembled and/or implanted, where the prosthetic heart valve has one or more leaflets with coaptation restriction features in the form of gathered portions, and one or more leaflets with coaptation restriction features in the form of leaflet portions being attached to other components of the prosthetic heart valve, or otherwise at least one prosthetic leaflet having both types of coaptation restriction features.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a frame; and
a plurality of prosthetic leaflets mounted within the frame;
wherein a first one of the plurality of prosthetic leaflets includes a fixation mechanism applied to the first one of the plurality of prosthetic leaflets, the fixation mechanism configured to transition from an initial condition at an initial time point to a subsequent condition at a subsequent time point after implantation,
wherein (i) when the fixation mechanism is in the initial condition, the fixation mechanism is configured to restrict mobility of the first one of the plurality of prosthetic leaflets to induce a first amount of regurgitation of blood through the plurality of prosthetic leaflets, and (ii) when the fixation mechanism is in the subsequent condition, the fixation mechanism is configured to induce a second amount of regurgitation of blood through the plurality of prosthetic leaflets, the second amount of regurgitation being less than the first amount of regurgitation.

2. The prosthetic heart valve of claim 1, wherein the fixation mechanism is configured to dissolve, degrade, absorb or otherwise release between the first time point and the subsequent time point after implantation to transition the fixation mechanism from the initial condition to the subsequent condition.

3. The prosthetic heart valve of claim 1, wherein the fixation mechanism is an adhesive.

4. The prosthetic heart valve of claim 1, wherein the fixation mechanism is a binding-type mechanism, agent, or material.

5. The prosthetic heart valve of claim 1, wherein the second amount of regurgitation is between about 0 mL and about 5 mL per heartbeat.

6. The prosthetic heart valve of claim 1, wherein the first one of the plurality of prosthetic leaflets includes a terminal free edge and an attached edge, a portion of the terminal free edge being folded over and attached, via the fixation mechanism, to a remaining portion of the first one of the plurality of prosthetic leaflets to form a modified free edge portion.

7. The prosthetic heart valve of claim 6, wherein in the initial condition of the fixation mechanism, the first one of the plurality of prosthetic leaflets has an initial effective axial height between the attached edge and the modified free edge portion, and in the subsequent condition of the fixation mechanism, the terminal free edge is no longer folded over and attached to the remaining portion of the first one of the plurality of prosthetic leaflets such that the first one of the plurality of prosthetic leaflets has a subsequent effective axial height between the attached edge and the terminal free edge, the subsequent effective axial height being greater than the initial effective axial height.

8. The prosthetic heart valve of claim 1, wherein, in the initial condition of the fixation mechanism, the first one of the plurality of prosthetic leaflets includes a gathered portion to which the fixation mechanism is applied to maintain the gathered portion in a gathered condition.

9. The prosthetic heart valve of claim 8, wherein in the initial condition of the fixation mechanism, the gathered portion extends generally orthogonal to a direction of blood flow through the prosthetic heart valve.

10. The prosthetic heart valve of claim 9, wherein in the initial condition of the fixation mechanism, the first one of the plurality of prosthetic leaflets has an initial effective axial height between an attached edge of the first one of the plurality of prosthetic leaflets and a free edge of the first one of the plurality of prosthetic leaflets, and in the subsequent condition of the fixation mechanism, the first one of the plurality of prosthetic leaflets has a subsequent effective axial height between the attached edge and the free edge, the subsequent effective axial height being greater than the initial effective axial height.

11. The prosthetic heart valve of claim 8, wherein in the initial condition of the fixation mechanism, the gathered portion extends generally parallel to a direction of blood flow through the prosthetic heart valve.

12. The prosthetic heart valve of claim 11, wherein in the initial condition of the fixation mechanism, a free edge of the first one of the plurality of prosthetic leaflets has an initial effective length and an initial tautness, and in the subsequent condition of the fixation mechanism, the free edge has a subsequent effective length and a subsequent tautness, the subsequent effective length being greater than the initial effective length, the initial tautness being greater than the subsequent tautness.

13. The prosthetic heart valve of claim 2, wherein, in the initial condition of the fixation mechanism, a portion of the first one of the plurality of prosthetic leaflets is attached, via the fixation mechanism, to (i) the frame and/or (ii) a skirt coupled to the frame.

14. The prosthetic heart valve of claim 13, wherein, in the initial condition of the fixation mechanism, the fixation mechanism prevents the portion of the first one of the plurality of prosthetic leaflets from moving toward remaining ones of the plurality of prosthetic leaflets, and in the subsequent condition of the fixation mechanism, the fixation mechanism does not prevent the portion of the first one of the plurality of prosthetic leaflets from moving toward remaining ones of the plurality of prosthetic leaflets.

15. The prosthetic heart valve of claim 14, wherein the portion of the first one of the plurality of prosthetic leaflets is a portion of a free edge of the first one of the plurality of prosthetic leaflets.

16. A prosthetic heart valve, comprising:
a frame; and
a plurality of prosthetic leaflets mounted within the frame;
wherein a first one of the plurality of prosthetic leaflets includes a first fixation mechanism having (i) an initial state in which the first fixation mechanism prevents the first one of the plurality of prosthetic leaflets from fully coapting with remaining ones of the plurality of prosthetic leaflets so that regurgitation of blood through the plurality of prosthetic leaflets is maintained in the initial state, and (ii) a subsequent state in which the first fixation mechanism does not prevent the first one of the plurality of prosthetic leaflets from fully coapting with the remaining ones of the plurality of prosthetic leaflets,
wherein the first one of the plurality of prosthetic leaflets includes a second fixation mechanism, the second fixation mechanism having (i) an initial state in which the second fixation mechanism prevents the first one of the plurality of prosthetic leaflets from fully coapting with remaining ones of the plurality of prosthetic leaflets so that regurgitation of blood through the plurality of prosthetic leaflets is maintained in the initial state, and (ii) a subsequent state in which the second fixation mechanism does not prevent the first one of the plurality of prosthetic leaflets from fully coapting with the remaining ones of the plurality of prosthetic leaflets, and
wherein when the first fixation mechanism and the second fixation mechanism are both in their respective subsequent states, regurgitation of blood through the plurality of prosthetic leaflets is less than when the first fixation mechanism and the second mechanism are both in their respective initial states.

17. The prosthetic heart valve of claim 16, wherein the first fixation mechanism is configured to dissolve, degrade, absorb or otherwise release over time to transition the first fixation mechanism from the initial state to the subsequent state, and the second fixation mechanism is configured to dissolve, degrade, absorb or otherwise release over time to transition the second fixation mechanism from the initial state to the subsequent state.

18. The prosthetic heart valve of claim 17, wherein the first fixation mechanism is configured to transition from the initial state to the subsequent state at a first time after implantation of the prosthetic heart valve, and the second fixation mechanism is configured to transition from the initial state to the subsequent state at a second time after implantation of the prosthetic heart valve, the first time being different than the second time.

19. The prosthetic heart valve of claim 18, wherein the first time is about one month, and the second time is about three months or about six months.

20. The prosthetic heart valve of claim 18, wherein the first time is about one month or about three months, and the second time is about six months.
